(19) United States
(12) Patent Application Publication
(10) Pub. No.: US 2006/0122282 A1
(43) Pub. Date: Jun. 8, 2006

(54) METHOD FOR TREATING SKIN DISORDERS WITH XANTHOPHYLLS

(76) Inventor: Todd Leonard, Minnetonka, MN (US)

Correspondence Address: SCHWEGMAN, LUNDBERG, WOESSNER & KLUTH, 121 S. 8TH STREET, SUITE 1600, MINNEAPOLIS, MN, 55402, US

(21) Appl. No.: 11/275,007

(22) Filed: Dec. 1, 2005

Related U.S. Application Data
(60) Provisional application No. 60/633,266, filed on Dec. 3, 2004.

(51) Int. Cl. A61K 31/12; A61K 31/015

(52) U.S. Cl. 514/690; 514/763

(57) ABSTRACT

The present invention provides for a method for treating a skin disorder in a mammal inflicted with a skin disorder. The present invention also provides for a method for retarding or reversing the loss of collagen fibers, abnormal changes in elastic fibers, or deterioration of small blood vessels in sundamaged mammalian skin. The present invention also provides for a method for exfoliating the skin surface of a mammal. The present invention also provides for a method for treating or preventing acne or a pimple in a mammal in need thereof. The methods include topically administering, to a mammal in need of such treatment, a composition that includes xanthophylls in a nontoxic amount, effective to treat the skin disorder.

### RELATED APPLICATIONS

The present application claims priority to U.S. Ser. No. 60/633,266 filed on 3 Dec. 2004, which is incorporated by reference herein, in its entirety.

### BACKGROUND OF THE INVENTION

Acne vulgaris is a chronic disorder of the pilosebaceous follicles (apparatus) characterized by comedones (blackheads), papules, pustules, cysts, nodules, and often scars, that appear on the most visible areas of the skin (e.g., the face, chest, back, neck, and upper arms). The pilosebaceous apparatus is largely under the control of endogenous hormones (mainly androgens) which are present in unusually high concentrations in the blood during adolescence and puberty, giving rise to an excessive production of sebum. The condition may worsen by a simultaneous increase in the rate of keratinization of the skin's horny layer (the stratum corneum). As the horny cells proliferate, they can form an occlusive plug or comedone which coupled with the increased production of the sebum, represents an ideal medium for the proliferation of the skin resident strains, such as the Gram positive anaerobic bacterium, Propionibacterium acnes. Eventually, the plugged follicles rupture and allow the discharge of their contents, causing local swelling and inflammation. The exposed follicles may darken from the deposition of pigment from damaged cells in the deeper layer of skin. In severe cases, acne can lead to hospitalization of the patient, extensive discomfort, and long term scarring of the skin.

There are numerous treatments available for treating acne. Typically, acne is treated with topical compositions in the form of creams, gels, emulsions or lotions that contain selected agents. These agents include hormones or hormone agonists and antagonists (EP A1 0 563 813 and U.S. Pat. No. 5,439,923), antimicrobial agents (U.S. Pat. No. 4,446,145, GB 2,088,717, GB 2,090,135, GB 1,054,124, U.S. Pat. No. 5,409,917), salicylic acid (U.S. Pat. No. 4,514,385, U.S. Pat. No. 4,355,028, EP A1 0 052 705, FR-A 2,581,542, and FR-A 2,607,498).

Oral administration of acne drugs is currently provided for severe cases of acne. These are reviewed in “Acne, A Review of Optimum Treatment” by Sykes N. I. and Webster G. F in Drugs 48, 59-70 (1994). Numerous side-effects have been described using oral administration of acne drugs. For example, isotretinoin, which is a derivative of vitamin A has associated risks of teratogenicity and may be a risk for women of childbearing age. Oral administration of antibiotics suited for treating acne may induce the appearance of adverse effects which include abdominal cramps, black tongue, cough, diarrhea, fatigue, irritation of the mouth and other undesirable symptoms.

Caucasians who have had a good deal of sun exposure in childhood will show the following gross cutaneous alterations in adult life: wrinkling, leatheriness, yellowing, looseness, roughness, dryness, mottling (hyperpigmentation) and various premalignant growths (often subclinical). These changes are most prominent in light-skinned persons who burn easily and tan poorly. The baleful effects of sunlight are cumulative, increasing with time often referred to as “photoaging”. Although the anatomic degradation of the skin is most advanced in the elderly, the destructive effects of excessive sun exposure are already evident by the second decade. Serious microscopic alterations of the epidermis and dermis occur decades before these become clinically visible. Wrinkling, yellowing, leatheriness, loss of elasticity are very late changes.

Retinoids (e.g. Vitamin A and its derivatives) are substances which are known to have a broad spectrum of biological activity. Most specifically, these substances affect cell growth, differentiation and proliferation. Retinoids affect the differentiation, maintenance, and proliferation of many types of cells whether they are of ectodermal, endodermal or mesodermal origin; whether they are epithelial, fibroblastic or mesenchymal; or whether they are neoplastic, preneoplastic or non-neoplastic. At present, retinoids have found clinical utility in the treatment of severe cystic acne, psoriasis, and other disorders of keratinization. Possible uses of retinoids are being explored in the prophylaxis and treatment of cancer. For a review of developments in retinoid therapy, see Pawson, B. A. et al. “Retinoids at the Threshold: Their Biological Significance and Therapeutic Potential”, Journal of Medicinal Chemistry 25:1269-1277 (1982).

The present status of retinoids in research and clinical medicine can be found in the publication of a symposium held in Geneva: J. H. Saurat. Editor, “Retinoids: New Trends in Research and Therapy,” Karger Publishing Co. (1985).

It is known to use certain retinoids, particularly vitamin A acid, topically for treatment of acne as set forth in my U.S. Pat. No. 3,729,568. Other known topical uses of vitamin A acid were reviewed by Thomas, J. R., et al, “The Therapeutic uses of Topical Vitamin A Acid”, Journal of American Academy of Dermatology 4:505-516 (1981) include, in addition to acne treatment, treatment of senile comedones, nevus comedonicus, linear verrucous nevus, plantar warts, pseudofollicultis, keratoacanthoma, solar keratosis of extremities, callosites, keratosis palmaris et plantaris. Darier's disease, ichthyosis, psoriasis, acanthosis nigricans, lichen planus, molluscum contagiosum, reactive perforating collagenosis, melasma, corneal epithelial abrasion, geographic tongue, Fox-Fordyce disease, cutaneous metastatic melanoma and keloids or hypertrophic scars.

It is believed that retinoids influence ultrastructural and proliferative properties of epidermal cells. However, these prior art uses of vitamin A acid have generally involved short term treatments in which relatively high concentrations retinoic acid are applied (i.e. sufficient to cause significant irritation and often peeling) in order to obtain a quick therapeutic effect of the particular condition, such as removal of comedones, as opposed to long-term treatment of normal aging or photographing skin.

U.S. Pat. Nos. RE36,068; 6,531,141 and 4,877,805 disclose compositions that include retinoids, useful to treat skin disorders. These disclosed compositions do not include xanthophylls.

FDA regulations (e.g., 21 C.F.R. Chapter 1, Section 333, Subpart D—Topical Acne Drug Products, Apr. 1, 2000 Edition) regulate what components (i.e., “active ingredients”), in a specified amount, may be described as treating acne (i.e., contains a topical acne drug). In order to follow FDA regulations, therefore, only a select number of active ingredients that are able to treat acne, in a specified amount, may be included in a composition when the composition is described as treating acne. Consequently, it is difficult to manufacture a composition that includes a topical acne drug, while at the same time maintaining (a) the solubility and stability of the active ingredients in the composition and (b) following FDA regulations.

There is a need therefore for new methods and composition for treating patients with skin disorders (e.g., acne), that have minimum adverse effects, have maximum efficacy, may be simple and comfortable to use, administers to the skin an effective and known amount of a topical acne drug, and complies with FDA regulations.

### SUMMARY OF THE INVENTION

The present invention provides for the cosmetic and pharmaceutical uses of compositions that include xanthophylls. The xanthophylls are present in the compositions in a known, discrete, safe, and effective amount. The compositions are useful to treat skin disorders (e.g., acne or photodamaged skin).

The present invention provides for a method for treating a skin disorder in a mammal inflicted with a skin disorder. The method includes topically administering, to a mammal in need of such treatment, a composition that includes xanthophylls in a nontoxic amount, effective to treat the skin disorder.

The present invention also provides for a method for retarding or reversing the loss of collagen fibers, abnormal changes in elastic fibers, or deterioration of small blood vessels in sundamaged mammalian skin. The method includes applying topically to the surface of the skin a composition that includes an effective and nontoxic amount of xanthophylls.

The present invention also provides for a method for exfoliating the skin surface of a mammal. The method includes applying topically to the surface of the skin a composition that includes an effective and nontoxic amount of xanthophylls.

The present invention also provides for a method for treating or preventing acne or a pimple in a mammal in need thereof. The method includes applying topically to the surface of the skin a composition that includes an effective and nontoxic amount of xanthophylls.

### DETAILED DESCRIPTION OF THE INVENTION

The present invention provides for the cosmetic and pharmaceutical uses of compositions that include xanthophylls. The xanthophylls are present in the compositions in a known, discrete, safe, and effective amount. The compositions are useful to treat skin disorders (e.g., acne or photodamaged skin).

References in the specification to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

It is appreciated that those of skill in the art understand that the terms used herein, unless expressly stated otherwise, include the singular as well as the plural. For example, the term “xanthophylls” includes the singular (i.e., one xanthophyll) as well as the plural (i.e., two or more different xanthophylls).

As used herein, a “topical acne drug” is a compound or combination of compounds that effectively prevents and/or treats acne or a pimple. Any suitable topical acne drug can be employed, provided the topical acne drug effectively treats and/or prevents acne or a pimple and the topical acne drug remains stable in the composition. Preferably, the stability is over a prolonged period of time, e.g., up to about 3 years, up to about 1 year, or up to about 6 months, typically experienced in the manufacturing, packaging, shipping, and/or storage of the composition.

Suitable topical acne drugs are disclosed, e.g., in Physician's Desk Reference (PDR), Medical Economics Company (Montvale, N.J.), (53rd Ed.), 1999; Mayo Medical Center Formulary, Unabridged Version, Mayo Clinic (Rochester, Minn.), January 1998; Merck Index, An Encyclopedia of Chemicals, Drugs, and Biologicals, (11th Ed.), Merck & Co., Inc. (Rahway, N.J.), 1989; and references cited therein. Suitable topical acne drugs include, e.g., salicylic acid, resorcinol, resorcinol acetate, calcipotriene, benzoyl peroxide, sulfur, retinol, retinoic acid, citric acid, an alpha hydroxy acid, retinal, pharmaceutically acceptable salts thereof, and combinations thereof. Preferably, the topical acne drug is salicylic acid, or a pharmaceutically acceptable salt thereof.

As used herein, “retinoid” refers to vitamin A or vitamin A-like compounds, including, but not limited to, retinoic acid (RA), a natural acidic derivative of vitamin A. Retinoids play a critical role in normal development, growth and differentiation by modulating the expression of target genes.

As used herein, “anthralin” refers to an anthraquinone (the 9, 10 quinone derivative of anthracene; anthraquinones can be made synthetically and also occur in naturally in aloe, cascara sagrada, senna, and rhubarb; the antineoplastic mitoxantrone is a synthetic derivative) derivative that reduces DNA synthesis and mitotic activity in hyperplastic epidermis, restoring the normal rate of epidermal cell proliferation and keratinization; used topically in the treatment of psoriasis and other skin conditions (also called dithranol).

As used herein, “coal tar” refers to a viscous black liquid containing numerous organic compounds that is obtained by the destructive distillation of coal. Coal tar can be distilled into many fractions to yield a number of useful organic products, including benzene, toluene, xylene, naphthalene, anthracene, and phenanthrene. These substances, called the coal-tar crudes, form the starting point for the synthesis of numerous products—notably dyes, drugs, explosives, flavorings, perfumes, preservatives, synthetic resins, and paints and stains. Coal tar is used medically to treat eczema, psoriasis, seborrheic dermatitis, and other skin disorders.

As used herein, “salicylic acid” refers to 2-hydroxybenzoic acid (C₆H₄(OH)CO₂H), which is a colorless, crystalline organic carboxylic acid. Salicylic acid is used to treat many skin disorders, such as acne, dandruff, psoriasis, seborrheic dermatitis of the skin and scalp, calluses, corns, common warts, and plantar warts.

As used herein, “photochemotherapy with ultraviolet A (PUVA)” refers to a type of ultraviolet radiation treatment (phototherapy) used for severe skin diseases. PUVA is a combination treatment which consists of Psoralen (P) administration and then exposure of the skin to long wave ultraviolet radiation (UVA). Psoralens include compounds which make the skin temporarily sensitive to UVA.

As used herein, “phototherapy with UVB” refers to a type of radiation treatment or therapy involving exposure to ultraviolet B light (wavelength 280-315 nm).

As used herein, “synergize” or “synergizes” or “synergistic” refers to the working together of two substances to produce an effect greater than the sum of their individual effects (www.webster-dictionary.org/definition/synergize.).

As used herein, “potentiate” or “potentiates” refers to the ability of one substance to make another substance (e.g., of one drug to make a second drug) effective or active or more effective or more active (http://www.ndif.org/Terms/potentiate.html).

As used herein, “alleviate” refers to a physical or mental lightening, lessening, eliminating or diminishing of the severity or length of time of a condition or symptom underlying the condition.

As used herein, “calcipotriene” refers to a synthetic topical form of vitamin D. It is involved in the growth and development of skin cells. Topical calcipotriene is used to treat plaque psoriasis (psoriasis with scaly patches). Chemically, calcipotriene is (5Z,7E,22E,24S)-24-cyclopropyl-9,10-secochola-5,7,10(19), 22-tetraene-1 alpha, 3 beta, 24-triol-, with the empirical formula C₂₇H₄₀O₃.

“Therapeutically effective amount” is intended to include an amount of, e.g., xanthophylls useful in the present invention, or an amount of a topical acne drug useful in the present invention, or an amount of the combination of compounds (e.g., xanthophylls and topical acne drug), e.g., to treat the acne or to treat the symptoms of the acne in a host. The combination of compounds is preferably a synergistic combination.

Synergy, as described for example by Chou and Talalay, Adv. Enzyme Regul., 22:27 (1984), occurs when the effect (e.g., treatment of skin disorder) of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased activity, or some other beneficial effect of the combination compared with the individual components.

As used herein, “acne” refers to an inflammatory follicular, papular, or pustular eruption involving the sebaceous apparatus. Acne is a disease of the skin where sebaceous glands are numerous (e.g., face, upper back, and chest) and characteristic lesions are present, e.g., open (blackhead) comedo, closed (whitehead) comedo, papule, pustule, or nodule. It is believed that acne results from the thickening of the follicular opening, increased sebum production, the presence of bacteria, or the host's inflammatory response. The types of acne include, e.g., acne conglobata, chloracne, and rosacea. See, e.g., Stedman's Medical Dictionary, 25th Ed., illustrated, Williams & Wilkins, Baltimore, Md., pp. 15-16 (1990) and Mosby's Medical, Nursing, & Allied Health Dictionary, (5th Ed.), Mosby: St. Louis, p. 19 (1998).

As used herein, a “pimple” refers to a small papule, pustule, or furnacle. See, e.g., Mosby's Medical, Nursing, & Allied Health Dictionary, (5th Ed.), Mosby: St. Louis, p. 1267 (1998).

As used herein, “fatty acids” refers to organic, monobasic acids derived from hydrocarbons by the equivalent of oxidation of a methyl group to an alcohol, aldehyde, and then acid. Fatty acids can be saturated, unsaturated, or partially unsaturated.

As used herein, “alfalfa” refers to lucem (Medicago sativa).

As used herein, “alpha carotene” refers to a compound of the formula:

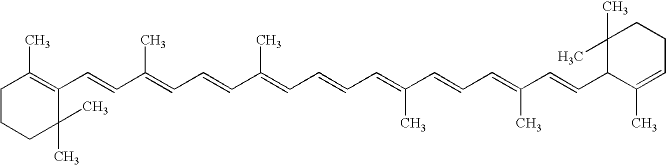

As used herein, “beta carotene” refers to a compound of the formula: 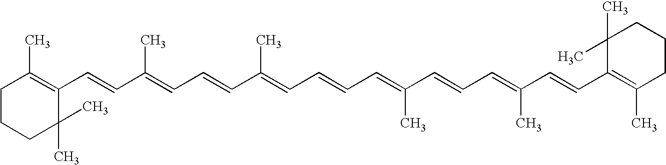
Beta-carotene is a proform (prodrug) of vitamin A, and is a lipid-soluble orange pigment found in many vegetables. Beta-carotene is converted to vitamin A in the body with an efficiency of approximately 50 percent.
As used herein, “vitamin A” refers to a compound of the formula: 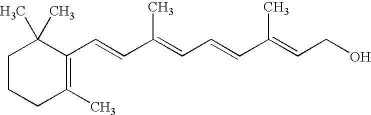
which is chemically designated as 3,7-dimethyl-9-(2,6,6-trimethyl-1-cyclohexen-1-yl)-2,4,6,8-nonatetraen-1-ol.

As used herein, “alleviate” refers to a physical or mental lightening, lessening, eliminating or diminishing of the severity or length of time of a condition or symptom underlying the condition.

As used herein, “chronic” refers to a condition, symptom or disease which persists over a long period of time and/or is marked by frequent recurrence (e.g., chronic colitis). Chronic disease refers to a disease which is of long continuance, or progresses slowly, in distinction from an acute disease, which quickly terminates.

As used herein, “skin disorder” refers to disorders of the skin including, but not limited to, disease of the skin, skin condition, skin disease, skin problems, which include, but are not limited to, acne, eczema, psoriasis, rosacea, skin cancer, skin burns, skin allergies, congenital skin disorders, acantholysis, acanthosis, acanthosis nigricans, dermatosis, disease, erythroderma, furunculosis, impetigo, jungle rot, keratoderma, keratodermia, keratonosis, keratosis, keratosis nigricans, leukoderma, lichen, livedo, lupus, melanism, melanosis, molluscum, necrobiosis lipoidica, necrobiosis lipoidica diabeticorum, pemphigus, prurigo, rhagades, Saint Anthony's fire, seborrhea, vitiligo, xanthoma, xanthosis, Psoriatic arthritis, Reiter's syndrome, Guttate psoriasis, Dyshidriotic eczema, Acute and chronic graft versus host disease, Systemic sclerosis, Morphea, Spongiotic dermatitis, Allergic dermatitis, Nummular eczema, Pityriasis rosacea, Pityriasis rubra pilaris, Pemphigus erythematosus, Pemphigus vulgaris, Lichenoid keratosis, Lichenoid nitidus, Lichen planus, Lichenoid dermatitis, Seborrheic dermatitis, Autosensitization dermatitis, Dermatitis herpetiformis, and Eosinophilic dermatitis. In one specific embodiment, the skin disorder can be mediated by an immunological response. In another specific embodiment, the skin disorder can be a lymphocyte-mediated skin disorder. In another specific embodiment, the skin disorder can be selected from the group of alopecia greata, psoriasis, atopic dermatitis, lupus erythematosis, bullous pemphigoid, psoriatic plaque, and combinations thereof. In another specific embodiment, the skin disorder can be psoriasis. In another specific embodiment, the skin disorder can be a chronic skin disorder. In another specific embodiment, the skin disorder can be an autoimmune skin disorder. In another specific embodiment, the skin disorder can be a malignant lymphoid disease that manifests in the skin.

As used herein, “cancer” includes a type of disease caused by cells that divide and grow uncontrollably, invading and disrupting other tissues and spreading to other areas of the body (metastasis). It is an abnormal uncontrolled growth of tissue that has potential to spread to distant sites of the body. Cancer exerts its deleterious effect on the body by: (a) Destroying the surrounding adjacent tissues: e.g. compressing nerves, eroding blood vessels, or causing perforation of organs; and (b) Replacing normal functioning cells in distant sites: e.g. replacing blood forming cells in the bone marrow, replacing bones leading to increased calcium levels in the blood, or in the heart muscles so that the heart fails. In suitable embodiments of the invention, the cancer is skin cancer.

As used herein, “skin cancer” refers to a cancer of the skin caused by contact with various chemical substances or by too much exposure to the sun or other sources of ultraviolet light. Skin cancers, the most common and most curable cancers, are also the most frequent secondary lesions in patients with cancer in other sites. Risk factors are a fair complexion, xeroderma pigmentosa, vitiligo, senile and seborrheic keratitis, Bowen's disease, radiation dermatitis, and hereditary basal cell nevus syndrome. The most common skin cancers are basal cell carcinomas and squamous cell carcinomas. Tumors of the sebaceous glands or sweat glands occur rarely. Basal cell carcinomas-typically raised, hard, reddish sores with a pearly surface-do not spread to other places in contrast to scaly, slightly raised squamous cell tumors that may become growths with widespread open sores and a nonhealing scab. Nonmelanomas (usually basal cell and squamous cell cancers) are the most common cancers of the skin. They are called nonmelanoma because they develop from skin cells other than melanocytes. Because they rarely spread elsewhere in the body, they are less worrisome than melanomas. Melanoma is a cancer that begins in the melanocytes. Because most of these cells keep on making melanin, melanoma tumors are often brown or black, but this is not always the case. Melanoma most often appears on the trunk of fair-skinned men and on the lower legs of fair-skinned women, but it can appear other places as well. While having dark skin lowers the risk of melanoma, it does not mean that a person with dark skin will never develop melanoma.

Basal cell carcinoma is the most common non-melanoma skin cancer. It begins in the lowest layer of the epidermis, called the basal cell layer. It usually develops on sun-exposed areas, especially the head and neck. Basal cell cancer is slow-growing and is not likely to spread to distant parts of the body.

Alpha-lipoic acid provides superior antioxidant protection due to the fact that it enhances the potency of other antioxidants in the body. Alpha-lipoic acid may be added to the composition of the present invention if desired, in any suitable and appropriate amount.

Phenolic compounds such oligomeric proanthocyanidins are additional useful antioxidants. Oligomeric proanthocyanidins are found naturally in grape seeds. Phenolic compounds may be added to the composition of the present invention if desired.

Antibiotic

The composition of the present invention can further include an antibiotic. As used herein, an “antibiotic” is any compound having activity against either Gram-positive or Gram-negative organisms (i.e., inhibits the growth or destroys the development of either Gram-positive or Gram-negative organisms). Stedman's Medical Dictionary, Illustrated, (25th Ed.), Williams & Wilkins: Baltimore (1990) and Mosby's Medical, Nursing, & Allied Health Dictionary, (5th Ed.), Mosby: St. Louis (1998).

Suitable antibiotics are disclosed, e.g., in Physician's Desk Reference (PDR), Medical Economics Company (Montvale, N.J.), (53rd Ed.), 1999; Mayo Medical Center Formulary, Unabridged Version, Mayo Clinic (Rochester, Minn.), January 1998; Merck Index, An Encyclopedia of Chemicals, Drugs, and Biologicals, (11th Ed.), Merck & Co., Inc. (Rahway, N.J.), 1989; University of Wisconsin Antimicrobial Use Guide, http://www.medsch.wisc.edu/clinsci/amcg/amcg.html; Introduction on the Use of the Antibiotics Guideline, Descriptions of Specific Antibiotic Classes, Thomas Jefferson University, http://jeffline.tju.edu/CWIS/OAC/antibiotics_guide/intro.html; and references cited therein.

Suitable antibiotics include, e.g., aminoglycosides, β-lactam antibiotics, cephalosporins, macrolides, miscellaneous antibiotics, penicillins, tetracyclines, antifungals, antimalarial agents, antituberculosis agents, antivirals, leprostatics, miscellaneous anti-infectives, quinolones, sulfonamides, urinary anti-infectives, nasal antibiotics, opthalmic antibiotics, opthalmic antivirals, opthalmic quinalones, opthalmic sulfonamides, skin and mucous membrane antibiotics, skin and mucous membrane antifungals, skin and mucous membrane antivirals, skin and mucous membrane miscellaneous anti-infectives, skin and mucous membrane scabicides and pedulicides, skin and mucous membrane antinepolasts, nitrofurans, and oxazolidinones. Physician's Desk Refernce (PDR), Medical Economics Company (Montvale, N.J.), (53rd Ed.), 1999 and Mayo Medical Center Formulary, Unabridged Version, Mayo Clinic (Rochester, Minn.), January 1998.

Aminoglycosides include, e.g., Amikacin (amikacin sulfate); Garamycin (gentamicin sulfate); Nebcin (tobramycin sulfate); Netromycin (netilmicin sulfate); Streptomycin Sulfate; and TOBI (tobramycin).

β-Lactam antibiotics include, e.g., Azactam (aztreonam); Cefotan (cefotetan); Lorabid (loracarbef); Mefoxin (cefoxitin); Merrem (meropenem); and Primaxin (imipenem and cilastatin for injectable suspension).

Cephalosporins include, e.g., Ancef (cefazolin); Ceclor (cefaclor); Cedax (ceftibuten); Cefizox (ceftizoxime sodium); Cefobid (cefoperazone sodium); Ceftin (cefuroxime axetil); Cefzil (cefprozil); Ceptaz (ceftazidime); Claforan (cefotaxime); Duricef (cefadroxil monohydrate); Fortaz (ceftazidime); Keflex (cephalexin); Keftab (cephalexin HCl); Kefurox (cefuroxime); Kefzol (cefazolin); Mandol (cefamandole nafate); Maxipime (cefepime HCl); Monocid (cefonicid sodium); Omnicef (cefdinir); Rocephin (ceftriaxone); Suprax (cefixime); Tazicef (ceftazidime); Tazidime (ceftazidime); Vantin (cefpodoxime proxetil); and Zinacef (cefuroxime).

Macrolides include, e.g., Biaxin (clarithromycin); Dynabac (dirithromycin); E.E.S. 200 (Erythromycin Ethylsuccinate); E.E.S. 400 (Erythromycin Ethylsuccinate); Ery-Ped 200 (Erythromycin Ethylsuccinate); EryPed 400 (Erythromycin Ethylsuccinate); Ery-Tab (Erythromycin delayed-release tablets); Erytbrocin Stearate (Erythromycin stearate); Ilosone (erythromycin estolate); PCE Dispertab (erythromycin particles in tablets); Pediazole (erythromycin ethylsuccinate and sulfisoxazole acetyl for oral suspension); Tao (troleandomycin); Zithromax (azithromycin); and Erythromycin.

Miscellaneous antibiotics include, e.g., Cleocin HCl (clindamycin hydrochloride); Cleocin Phosphate (clindamycin phosphate); Coly-Mycin M (colistimethate sodium); and Vancocin HCl (vancomycin hydrochloride).

Penicillins include, e.g., Amoxil (amoxicillin); Augmentin (amoxicillin/clavulanate potassium); Bicillin C-R 900/300 (Penicillin G benzathine and Penicillin G procaine suspension); Bicillin C-R (Penicillin G benzathine and Penicillin G procaine suspension); Bicillin L-A (Penicillin G benzathine suspension); Geocillin (carbencillin indanyl sodium); Mezlin (sterile mezlocillin sodium); Omnipen (ampicillin); Pen-Vee K (penicillin V potassium); Pfizerpen (penicillin G potassium); Pipracil (piperacillin sodium); Spectrobid (bacampicillin HCl); Ticar (ticarcillin disodium); Timentin (ticarcillin disodium and clavulanate potassium); Unasyn (ampicillin sodium/sulbactam sodium); Zosyn (piperacillin sodium and tazobactam sodium); and Dicloxacillin Sodium.

Tetracyclines include, e.g., Achromycin V (tetracycline HCl); Declomycin (demeclocycline HCl); Dynacin (minocylcine HCl); Minocin (minocycline hydrochloride); Monodox (Doxycycline monohydrate capsules); Terramycin (oxytetracyline); Vectrin (minocycline hydrochloride); Vibramycin Calcium (doxycycline sodium); Vibramycin Hyclate (doxycycline hyclate); Vibramycin Monohydrate (doxycycline monohydrate); Vibra-Tabs (doxycycline hydrate); Declomycin (demeclocycline HCl); Vibramycin (doxycycline); Dynacin (Minocyline HCl); Terramycin (oxytetracycline HCl); Achromycin V capsules (tetracycline HCl); Lincomycins; and Cleocin HCl (clindamycin HCl).

Antifungals include, e.g., Abelcet (amphotericin B lipid complex); AmBisome (amphotericin B); Amphotec (amphotericin B cholesterol sulfate complex); Ancobon (flucytosine); Diflucan (fluconazole); Fulvicin P/G (ultramicrosize griseofulvin); Fulvicin P/G 165 and 330 (ultramicrosize griseofulvin); Grifulvin V (griseofulvin); Gris-PEG (griseofulvin ultramicrosize); Lamisil (terbinafine hydrochloride); Nizoral (ketoconazole); Amphotericin B; Lotrimin (clotrimazole); Dapsone tablets (dapsone); Diflucan (fluconazole); Monistat-Derm cream (miconazole); Mycostatin Cream (nystatin); and Sporanox (itraconazole).

Antimalarial agents include, e.g., Aralen hydrochloride (chloroquine HCl); Aralen phosphate (chloroquine phosphate); Daraprim (pyrimethamine); Lariam (mefloquine HCl); and Plaquenil (hydroxychloroquine sulfate).

Antituberculosis agents include, e.g., Capastat sulfate (capreomycin sulfate); Myambutol (ethambutol hydrochloride); Mycobutin (rifabutin capsules); Nydrazid (isoniazid injection); Paser (aminosalicylic acid); Priftin (rifapentine); Pyrazinamide tablets (pyrazinamide); Rifadin (rifampin capsules); Rifadin IV (rifampin for injection); Rifamate (rifampin and isoniazid); Rifater (rifampin, isoniazid and pyrazinamide); Seromycin (cycloserine capsules); Streptomycin Sulfate; Tice BCG (BCG vaccine); Cycloserine (seromycin capsules); Urised (Methenamine); and Trecator-SC (ethionamide tablets).

Antivirals include, e.g., Alferon N (interferon alfa-n3); Crixivan (indinavir sulfate); Cytovene (ganciclovir); Cytovene-IV (ganciclovir sodium); Epivir (lamivudine); Famvir (famciclovir); Flumadine (rimantadine HCl); Foscavir (foscamet sodium); Hivid (zalcitabine); Intron A (interferon alfa-2b); Invirase (saquinavir mesylate); Norvir (ritonavir); Rebetron combination therapy, which contains Rebetrol (ribavirin) and Intron A (inteferon alfa-2b); Rescriptor (delavirdine mesylate); Retrovir (ziduvudine); Retrovir IV (zidovudine); Symmetrel (amantadine hydrochloride); Synagis (palivizumab); Valtrex (valacyclovir HCl); Videx (didanosine); Viracept (nelfinavir mesylate); Viramune (nevirapine); Virazole (ribavirin); Vistide (cidofovir); Zerit (stavudine (d4T)); Symmetrel Syrup (amantadine HCl); Combivir Tablets (lamiduvine); and Zovirax (acyclovir).

Leprostatics include, e.g., Dapsone Tablets (dapsone).

Miscellaneous anti-infectives include, e.g., Daraprim (pyrimethamine); Flagyl 375 (metronidazole); Flagyl ER Tablets (metronidazole); Flagyl I.V. (metronidazole); Furoxone (furazolidone); Mepron (atovaquone); and Neutrexin (trimetrexate glucuronate).

Quinolones include, e.g., Cipro (ciprofloxacin HCl); Floxin (ofloxacin); Levaquin (levofloxacin); Mazaquin (lomefloxacin HCl); Noroxin (norfloxacin); Penetrex (enoxacin); Raxar (grepafloxacin HCl); Trovan (trovafloxacin mesylate); and Zagam (sparfloxacin).

Sulfonamides include, e.g., Bactrim (trimethoprim and sulfamethoxazole); Bactrim DS (trimethoprim and sulfamethoxazole double strength); Pediazole (erythromycin ethylsuccinate and sulfisaxazole acetyl); Septra (trimethoprim and sulfamethoxazole); Septra DS (trimethoprim and sulfamethoxazole); Co-Trimoxazole, Sulfadiazine, Bactrim I.V. Infusion (sulfamethoxazole); Sulfapyridine, and Pediazole (erythromycin ethylsuccinate and sulfisoxazole acetyl).

Urinary anti-infectives include, e.g., Furadantin (nitrofurantoin); Macrobid (nitrofurantoin monohydrate macrocrystals); Macrodantin (nitrofurantoin macrocrystals); Monurol Sachet (fosfomycin tromethamine); NegGram Caplets (nalidixic acid); Septra (trimethoprim and sulfamethoxazole); Septra DS (trimethoprim and sulfamethoxazole); Urised (a combination of the antiseptics methenamine, methylene blue, phenyl salicylate, benzoic acid and parasympatholytics (atropine sulfate) hyoscyamine); Urobiotic-250 Capsules (oxytetracycline HCl, sulfamethizole and phenazopyridine HCl); and Uroqid Acid No. 2 Tablets (methenamine mandelate).

Nasal antibiotics include, e.g., Bactroban (mupirocin).

Opthalmic antibiotics include, e.g., Chloromycetin opthalmic (chloramphenical); Cortisporin (neomycin and polymyxin β sulfates and hydrocortisone acetate cream); Ilotycin (erythromycin opthalmic ointment); NeoDecadron (neomycin sulfate—dexamethasone sodium phosphate); Polytrim (trimethoprim and polythyxin β sulfate opthalmic solution); Terra-Cortril (oxytetracycline HCl and hydrocortisone acetate); Terramycin (oxytetracycline); and TobraDex (tobramycin and dexamethasone opthalmic suspension and ointment).

Opthalmic antivirals include, e.g., Vira-A opthalmic ointment, (vidarabine).

Opthalmic quinalones include, e.g., Chibroxin (norfloxacin opthalmic solution); Ciloxan opthalmic solution, (Ciprofloxacin HCl); Ciloxan opthalmic ointment, (Ciprofloxacin HCl); and Ocuflox opthalmic solution (ofloxacin).

Opthalmic sulfonamides include, e.g., Blephamide opthalmic ointment (sulfacetamide sodium and prednisolone acetate); and Blephamide opthalmic suspension (sulfacetamide sodium and prednisolone acetate).

Skin and mucous membrane antibiotics include, e.g., A/T/S (erythromycin); Bactroban (mupirocin); Benzamycin (erythromycin-benzoyl peroxide topical gel); Betadine (povidone-iodine); Cleocin T (clindamycin phosphate topical solution); Clindets (clindamycin phosphate pledgets); Cortisporin (neomycin, polymyxin B sulfates and hydrocortisone acetate cream); Emgel (erythromycin); Erycette (erythromycin topical solution); Garamycin (gentamicin sulfate); Klaron (sodium sulfacetamide lotion); Mycostatin (nystatin cream); Theramycin Z (erythromycin topical solution); T-Stat (erythromycin); Chloromycetin (chloramphenicol opthalmic ointment); Cortisporin (neomycin and polymyxin B sulfates, bacitracin zinc and hydrocortisone opthalmic ointment); Ilotycin (erythromycin); NeoDecadron (neomycin sulfate-dexamethasone sodium phosphate); Polytrim (trimethoprim and polymyxin B sulfate); Terra-Cortril (oxytetracycline HCl and hydrocortisone acetate); Terramycin (oxytetracycline); and TobraDex (tobramycin and dexamethasone opthalmic suspension and ointment).

Skin and mucous membrane antifungals include, e.g., Exelderm (sulconazole nitrate); Fungizone (amphotericin B oral suspension); Lamisil (terbinafine hydrochloride cream); Loprox (ciclopiroxolamine); Lotrimin (clotrimazole); Lotrisone (clotrimazole and betamethasone diproprionate); Mentax (butenafine HCl); Monistat-Derm (miconazole nitrate); Mycelex (clotrimazole); Mycostatin (nystatin); Naftin (naftifine HCl); Nizoral (ketoconazole); Nystop (nystatin); Oxistat (oxiconazole nitrate); Selsun Rx (2.5% selenium sulfide lotion); and Spectazole (econazole nitrate).

Skin and mucous membrane antivirals include, e.g., Denavir (penciclovir cream); and Zovirax (acyclovir).

Skin and mucous membrane miscellaneous anti-infectives include, e.g., Benzashave (benzoyl peroxide); Betadine (povidone-iodine); Betasept (chlorhexidine gluconate); Cetaphil (soap substitute); Clorpactin WCS-90 (sodium oxychlorosene); Dapsone Tablets (dapsone); Desquam-E (benzoyl peroxide); Desquam-X (benzoyl peroxide); Hibiclens (chlorhexidine gluconate); Hibistat (chlorhexidine gluconate); Impregon (tetrachlorosalicylanilide 2%); MetroCream (metronidazole); MetroGel (metronidazole); Noritate (metronidazole); pHisoHex (hexachlorophene detergent cleanser); Sulfacet-R (sodium sulfacetamide 10% and sulfur 5%); Sulfamylon (matenide acetate); Triaz (benzoyl peroxide); and Vanoxide-HC (benzoyl peroxide hydrocortisone).

Skin and mucous membrane scabicides and pedulicides include, e.g., Acticin (permethrin); Elimite (permethrin); Eurax (crotamiton); and Lindane Lotion USP 1% (lindane).

Skin and mucous membrane antinepolasts include, e.g., Efudex (fluorouracil); and Fluoroplex (fluorouracil).

Nitrofurans include, e.g., Furadantin Oral Suspension (nitrofurantoin).

Oxazolidinones include, e.g., Zyvox (linezolid).

It is appreciated that those skilled in the art understand that the antibiotic useful in the present invention is the biologically active compound present in any of the antibiotic formulations disclosed above. For example, Azactam (aztreonam) is typically available as an injectable solution. The antibiotic, however, is (z)-2-[[[(2-amino-4-thiazolyl) [[(2S,-3S)-2-methyl-4-oxo-1-sulfo-3-azetidinyl] carbamoyl] methylene] amino] oxy]-2-methyl propionic acid. Physician's Desk Reference (PDR), Medical Economics Company (Montvale, N.J.), (53rd Ed.), pp. 820-823, 1999.

Amikacin (amikacin sulfate) is commercially available from Elkins-Sinn and is D-Streptamine, O-3-amino-3-deoxy-α-D-glucopyranosyl-(1→6)-O-6-deoxy-α-D-glucopyranosyl-(1→4)]-N′-(4-amino-2 hydroxy-1-oxobutyl)-2-deoxy-, (S)-, sulfate (1:2) (salt).

Garamycin (gentamicin sulfate) is commercially available from Schering.

Nebcin (tobramycin sulfate) is commercially available from Lilly and is 0-3-amino-3-deoxy-α-D-glucopyranosyl-(1→4)-O-[2,6-diamino-2,3-6-trideoxy-α-D-ribo-hexopyranosyl-(1→6)]-2-deoxy-L-streptamine, sulfate (2:5) (salt).

Netromycin (netilmicin sulfate) is commercially available from Schering and is O-3-Deoxy-4-C-methyl-3-(methylamino)-β-L-ara-binopyranosyl (1→4)-O-[2,6-diamino-2,3,4,6-tetradeoxy-α-D-glycero-hex-4-enopyranosyl-(1→6)-2-deoxy-N-3-ethyl-L-streptamine sulfate (2:5) salt.

Streptomycin Sulfate is commercially available from Pfizer and is D-Streptamine, O-2-deoxy-2-(methyl amino)-α-L-glucopyranosyl-(1→2)-O-5-deoxy-3-C-formyl-α-L-lyxofuranosyl, (1→4)-N,N′-bis(aminoiminomethyl)-, sulfate (2:3) (salt).

TOBI (tobramycin) is commercially available from Pathogenesis Corporation and is O-3-amino-3-deoxy-α-D-glucopyranosyl-(1→4)-O-[2,6-diamino-2,3,6-trideoxy-α-D-ribo-hexopyranosyl-(1→6)]-2-deoxy-L-streptamine.

Azactam (aztreonam) is commercially available from Bristol-Myers Squibb and is (Z)-2-[[[(2-amino-4-thiazolyl) [[(2S,-3 S)-2-methyl-4-oxo-1-sulfo-3-azetidinyl] carbamoyl] methylene] amino] oxy]-2-methylpropionic acid.

Cefotan (cefotetan) is commercially available from Zeneca and is the disodium salt of [6R-(6a,7a)]-7-[[[4-(2-amino-1-carboxy-2-oxoethylidene)-1-3, dithietan-2-yl] carbonyl] amino]-7-methoxy-3-[[(1-methyl-1H-tetrazol-5-yl) thio] methyl]-8-oxo-5-thia-1-azabicyclo [4.2.0] oct-2-ene-2-carboxylic acid.

Lorabid (loracarbef) is commercially available from Lilly and is (6R,7S)-7-[(R)-2-amino-2-phenylacetamido]-3-chloro-8-oxo-1-azabicyclo [4.2.0] oct-2-ene-2-carboxylic acid, monohydrate.

Mefoxin (cefoxitin) is commercially available from Merck and is sodium (6R,7S)-3-(hydroxymethyl)-7-methoxy-8-oxo-7-[2-(2-thienyl) acetamido]-5-thia-1-azabicylo [4.2.0] oct-2-ene-2-carboxylate carbamate (ester).

Merrem (meropenem) is commercially available from Zeneca and is (4R,5S, 6S)-3-[(3S,5S)-5-(Dimethylcarbamoyl)-3-pyrrolidinyl] thiol]-6-[(1R)-1-hydroxyethyl]-4-methyl-7-oxo-1-azabicyclo [3.2.0] hept-2-ene-2-carboxylic acid trihydrate.

Primaxin (imipenem and cilastatin for injectable suspension) is commercially available from Merck and is (1) imipenem is N-formimidoylthienamycin monohydrate, chemical name is [5R-[5α,6α(R*)]]-6-(1-hydroxyethyl)-3-[[2-[(iminomethyl) amino] ethyl] thio]-7-oxo-1-azabicylco [3.2.0]hept-2-ene-2-carboxylic acid monohydrate, cilastatin sodium is [R-[R*,S*,-(Z)]]-7-[(2-amino-2-carboxyethyl) thio]-2-[[(2,2-dimethyl cyclopropyl) carbonyl] amino]-2-heptenoic acid, monosodium salt.

Ancef (cefazolin) is commercially available from SmithKline Beecham and is 3-{[(5-methyl-1,3,4-thiadiazol-2-yl) thio-methyl]}-8-oxo-7-[2-(1H-tetrazol-1-yl) acetamido]-5-thia-1-azabicyclo [4.2.0] oct-2-ene-2-carboxylic acid.

Ceclor (cefaclor) is commercially available from Lilly and is 3-chloro-7-D-(2-phenylglycinamido)-3-cephem-4-carboxylic acid monohydrate; Cedax (ceftibuten) is commercially available from Schering and is (+)-(6R,7R)-7-[(Z)-2-(2-(2-amino-4-thiazoly)-4-carboxycrotonamido]-8-oxo-5-thia-1-azabicyclo [4.2.0] oct-2-ene-2-carboxylic acid, dihydrate.

Cefizox (ceftizoxime sodium) is commercially available from Fujisawa and is sodium salt of [6R-[6α,7β(Z)]]-7 [[2,3, dihydro-2-imino-4-thiazolyl)(methoxy amino) acetyl] amino]-8-oxo-5-thia-1-azabicyclo [4.2.0] oct-2-ene-2-carboxyolic acid.

Cefobid (cefoperazone sodium) is commercially available from Pfizer and is sodium (6R,7R)-7 [(R)-2-(4-ethyl-2,3, dioxo-1-piperazine carboxamido)-2-(p-hydroxyphenyl)-acetamido)-3-[[(1-methyl-1H-tetrazol-5-yl) thio] methyl]-8-oxo-5-thia-1-azabicyclo [4.2.0] oct-2-ene-2-carboxylate.

Ceftin (cefuroxime axetil) is commercially available from Glaxo Wellcome and is (RS)-1-hydroxyethyl (6R,7R)-7-[2-(2-furyl) glyoxylamido]-3-(hydroxyethyl)-(8-oxo-5-thia-1-aza-bicyclo [4.2.0] oct-2-ene-2-carboxylate, 72 (Z)-O-methyl-oxime), 1-acetate 3-carbamate.

Cefzil (cefprozil) is commercially available from Bristol-Myers Squibb and is (6R,7R)-7-[(R)-2-amino-2-(p-hydroxyphenyl) acetamido]-8-oxo-3-propenyl-5-thia-1-azabicyclo [4.2.0] oct-2-ene-2-carboxylic acid monohydrate.

Ceptaz (ceftazidime) is commercially available from Glaxo Wellcome and is 1-[[7-[[(2-amino-4-thiazolyl) [(1-carboxy-1-methylethoxy) imine] acetyl] amino]-2-carboxy-8-oxo-5-thia-1-azabicyclo [4.2.0] oct-2-en-3-yl] methyl]-, hydroxide, inner salt, [6R-[6α,7β(Z)]].

Claforan (cefotaxime) is commercially available from Hoescht Marion Roussel and is 7-[2-(2-amino-4-thiazolyl) glyoxylamido]-3-(hydroxy methyl)-8-oxo-5-thia-1-azabicyclo [4.2.0] oct-2-ene-2-carboxylate 72 (Z)-(O-methyloxime), acetate (ester).

Duricef (cefadroxil monohydrate) is commercially available from Bristol-Myers Squibb and is 5-Thia-1-azabicyclo [4.2.0] oct-2-ene-2-carboxylic acid, 7-[[amino (4-hydroxyphenyl) acetyl] amino]-3-methyl-8-oxo,-monohydrate, [6R-[6α,7β(R*)]]-.

Fortaz (ceftazidime) is commercially available from Glaxo Wellcome and is 1-[[7-[[(2-amino-4-thiazolyl) [1-carboxy-1-methylethoxy) imino] acetyl] amino]-2-carboxy-8-oxo-5-thia-1-azabicyclo [4.2.0] oct-2-en-3-yl] methyl]-, hydroxide, inner salt [6R-[6α,7β(Z)]].

Keflex (cephalexin) is commercially available from Dista and is 7-(D-α-Amino-α-phenyl acetamido)-3-methyl-3-cephem-4-carboxylic acid monohydrate.

Keftab (cephalexin HCl) is commercially available from Dura and is 7-(D-2-Amino-2-phenylacetamido)-3-methyl-3-cephem-4-carboxylic acid hydrochloride monohydrate.

Kefurox (cefuroxime) is commercially available from Lilly and is the sodium salt of (6R.7R) 3-carbamoyloxymethyl-7-[Z-2-methoxyimino-2-(fur-2-yl) acetamiodo] ceph-3-em-4-carboxylate.

Kefzol (cefazolin) is commercially available from Lilly and is the sodium salt of 3-{[(5-methyl-1,3,4-thiadiazol-2-yl) thio] methyl}-8-oxo-7-[2-(1H-tetrazol-1-yl) acetamido]-5-thia-1-azabicyclo [4.2.0] oct-2-ene-2-caboxylic acid.

Mandol (cefamandole nafate) is commercially available from Lilly and is 5-thia-1-azabicyclo [4.2.0] oct-2-ene-2-carboxylic acid, 7-[[(formyloxy) phenyl acetyl] amino]-3-[[(1-methyl-1H-tetrazol-5-yl) thio] methyl]-8-oxo-, mono-sodium salt, [6R-[6α-7β(R*)]].

Maxipime (cefepime HCl) is commercially available from Bristol-Myers Squibb and is 1-[[6R,7R)-7-[2-amino-4-thiazolyl)-glyoxylamido]-2-carboxy-8-oxo-5-thia-1-azabicyclo-[4.2.0]oct-2-en-3-yl] methyl]-1-methylpyrrolidinium chloride, 72-(Z)-(O-methyloxime), monohydrochloride, monohyrate.

Monocid (cefonicid sodium) is commercially available from SmithKline Beecham and is 5-Thia-1-azabicyclo [4.2.0] oct-2-ene-2-carboxylic acid, 7-[(hydroxyphenyl-acetyl)-amino]-8-oxo-3-[[1-(sulfomethyl)-1H-tetrazol-5-yl] thio] methyl]-disodium salt, [6R-[6α,7β(R*)]].

Omnicef (cefdinir) is commercially available from Parke Davis and is [6R-[6α,7β(Z)]]-7-[[(2-amino-4-thiazolyl)-(hydroxyimino) acetyl] amino]-3-ethenyl-8-oxo-5-thia-1-azabicyclo [4.2.0] oct-2-ene-2-carboxylic acid.

Rocephin (ceftriaxone) is commercially available from Roche Laboratories and is (6R,7R)-7-[2-(2-Amino-4-thiazolyl) glyoxylamido]-8-oxo-3-[[(1,2,5,6-tetrahydro-2-methyl-5,6-dioxo-as-triazin-3-yl) thio] methyl]-5-thia-1-azabicyclo [4.2.0] oct-2-ene-2-carboxylic acid, 72-(Z)-O-methyloxime), disodium salt, sesquaterhydrate.

Suprax (cefixime) is commercially available from Lederle Laboratories and is (6R,7R)-7-[2-(2-Amino-4-thiazolyl) glyoxylamido]-8-oxo-3-vinyl-5-thia-1-azabicyclo [4.2.0] oct-2-ene-2-carboxylic acid, 72-(Z)-[O-(carboxymethyl) oxime] trihydrate.

Tazicef (ceftazidime) is commercially available from SmithKline Beecham and is pentahydrate of Pyridinium, 1-[[7-[[2-amino-4-thiazolyl) [(1-carboxy-1-methylethoxy) imino] acetyl] amino]-2-carboxy-8-oxo-5-thia-1-azabicyclo (4.2.0) oct-2-en-3-yl] methyl]-hydroxide, inner salt, [6R,-[6α,7β(Z)]].

Tazidime (ceftazidime) is commercially available from Lilly and is pentahydrate of Pyridinium, 1-[[7-[[2-amino-4-thiazolyl) [(1-carboxy-1-methylethoxy) imino] acetyl] amino]-2-corboxy-8-oxo-5-thia-1-azabicyclo (4.2.0) oct-2-en-3-yl] methyl]-hydroxide, inner salt, [6R,-[6α,7β(Z)]].

Vantin (cefpodoxime proxetil) is commercially available from Pharmacia & Upjohn and is (RS)-1 (isopropoxycarbonyloxy) ethyl (+)-(6R,7R)-7-[2-(2-amino-4-thiazolyl)-2-{(Z)methoxyimino} acetamido]-3-methoxymethyl-8-oxo-5-thia-1-azabicyclo [4.2.0] oct-2-ene-2-carboxylate.

Zinacef (cefuroxime) is commercially available from Glaxo Wellcome and is sodium salt of (6R,7R)-3-carbamoyloxymethyl-7-[Z-2-methoxy-imino-2-fur-2-yl) acetamido] ceph-3-em-4-carboxylate.

Biaxin (clarithromycin) is commercially available from Abbott and is 6-O-methylerythromycin.

Dynabac (dirithromycin) is commercially available from Sanofi and is (9S)-9-Deox-11-deoxy-9,11-[imino [(1R)-2-(2-methoxyethoxy)-ethylidene] oxy] erythromycin.

E.E.S. 200 (Erythromycin Ethylsuccinate) is commercially available from Abbott and is erythromycin 2′-(ethylsuccinate).

E.E.S. 400 (Erythromycin Ethylsuccinate) is commercially available from Abbott and is erythromycin 2′-(ethylsuccinate).

Ery-Ped 200 (Erythromycin Ethylsuccinate) is commercially available from Abbott and is erythromycin 2′-(ethylsuccinate).

EryPed 400 (Erythromycin Ethylsuccinate) is commercially available from Abbott and is erythromycin 2′-(ethylsuccinate).

Ery-Tab (Erythromycin delayed-release tablets) is commercially available from Abbott and is (3R*,4S*,5S*,6R*,7R*,9R*,11R*,12R*,13S*,14R*)-4-[(2,6-dideoxy-3-C-methyl-3-O-methyl-α-L-ribo-hexopyranosyl) oxy]-14-ethyl-7,12,13-trihydroxy-3,5,7,9,11,13, hexamethyl-6-[[3,4,6-trideoxy-3-(dimethylamino)-β-D-xylo-hex-opyranosyl] oxy] oxacyclotetradecane-2,10-dione.

Erythrocin Stearate (Erythromycin stearate) is commercially available from Abbott and is the stearate salt of (3R*,4S*,5S*,6R*,7R*,9R*,11R*,12R*,13S*,14R*)-4-[(2,6-dideoxy-3-C-methyl-3-O-methyl-α-L-ribo-hexopyranosyl) oxy]-14-ethyl-7,12,13-trihydroxy-3,5,7,9,11,13, hexamethyl-6-[[3,4,6-trideoxy-3-(dimethylamino)-β-D-xylo-hex-opyranosyl] oxy] oxacyclotetradecane-2,10-dione.

Ilosone (erythromycin estolate) is commercially available from Dista and is erythromycin 2′-propionate, dodecyl sulfate.

PCE Dispertab (erythromycin particles in tablets) is commercially available from Abbott and is (3R*,4S*,5S*,6R*,7R*,9R*,11R*,12R*,13S*,14R*)-4-[(2,6-dideoxy-3-C-methyl-3-O-methyl-α-L-ribo-hexopyranosyl) oxy]-14-ethyl-7,12,13-trihydroxy-3,5,7,9,11,13, hexamethyl-6-[[3,4,6-trideoxy-3-(dimethylamino)-β-D-xylo-hex-opyranosyl] oxy] oxacyclotetradecane-2,10-dione.

Pediazole (erythromycin ethylsuccinate and sulfisoxazole acetyl for oral suspension) is commercially available from Ross Products and is 2′-ethylsuccinyl ester of erythromycin (erythromycin ethylsuccinate) and N-(3,4-dimethyl-5-isoxazolyl)-N-sulfanilylacetamide (sulfisoxazole acetyl).

Tao (troleandomycin) is commercially available from Pfizer and is the synthetically derived acetylated ester of oleandomycin.

Zithromax (azithromycin) is commercially available from Pfizer and is (2R,3S,4R,5R,8R,10R,11R,12S,13S,14R)-13-[(2,6-dideoxy-3-C-methyl-3-O-methyl-α-L-ribo-hexopyranosyl) oxy]-2-ethyl-3,4,10-trihydroxy-3,5,6,8,10,12,14-heptamethyl-11-[[3,4,6-trideoxy-3-(dimethylamino)-β-D-xylo-hexopyranosyl] oxy]-1-oxa-6-azacyclopentadecan-15-one.

Erythromycin, which is (3R*,4S*,5S*,6R*,7R*,9R*,11R*,12R*,13S*,14R*)-4-[(2,6-dideoxy-3-C-methyl-3-O-methyl-α-L-ribo-hexopyranosyl) oxy]-14-ethyl-7,12,13-trihydroxy-3,5,7,9,11,13, hexamethyl-6-[[3,4,6-trideoxy-3-(dimethylamino)-β-D-xylo-hex-opyranosyl] oxy] oxacyclotetradecane-2,10-dione.

Cleocin HCl (clindamycin hydrochloride) is commercially available from Pharmacia & Upjohn and is the hydrated hydrochloride salt of clindamycin, a semisynthetic antibiotic produced by a 7 (S)-chlorosubstitution of the (7R) hydroxyl group of lincomycin.

Cleocin Phosphate (clindamycin phosphate) is commercially available from Pharmacia & Upjohn and is L-threo-α-D-galacto-Octopyranoside, methyl 7 chloro-6, 7, 8,-trideoxy-6-[[(1-methyl-4-propyl-2-pyrrolidinyl) carbonyl] amino]-1-thio-, 2-(dihydrogen phosphate), (2S-trans)-.

Coly-Mycin M (colistimethate sodium) is commercially available from Monarch.

Vancocin HCl (vancomycin hydrochloride) is commercially available from Lilly.

Amoxil (amoxicillin) is commercially available from SmithKline Beecham and is (2S,5R,6R)-6-[(R)-(−)-2-amino-2-(p-hydroxyphenyl) acetamido]-3,3-dimethyl-7-oxo-4-thia-1-azabicyclo [3.2.0]-heptane-2-carboxylic acid trihydrate.

Augmentin (amoxicillin/clavulanate potassium) is commercially available from SmithKline Beecham and is (2S,5R,6R)-6-[(R)-(−)-2-amino-2-(p-hydroxy phenyl) acetamido]-3,3-dimethyl-7-oxo-4-thia-1-azabicyclo [3.2.0]-heptane-2-carboxylic acid trihydrate (amoxicillin) and potassium (Z)-(2R,5R)-3-(2-hydroxyethylidene)-7-oxo-4-oxa-1-azabicyclo [3.2.0]-heptane-2-carboxylate (clavulanate potassium).

Bicillin C-R 900/300 (Penicillin G benzathine and Penicillin G procaine suspension) is commercially available from Wyeth-Ayerst and is (2S,5R,6R)-3,3-Dimethyl-7-oxo-6-(2-phenylacetamido)-4-thia-1-azabicyclo [3.2.0]heptane-2-carboxylic acid compound with N,N′-dibenzylethylenediamine (2:1), tetrahydrate (Penicillin G benzathine) and (2S,5R,6R)-3,3-Dimethyl-7-oxo-6-(2-phenylacetamido)-4-thia-1-azabicyclo [3.2.0]heptane-2-carboxylic acid compound with 2-(diethylamino) ethyl p-amino benzoate compound (1:1) monohydrate (Penicillin G procaine).

Bicillin C-R (Penicillin G benzathine and Penicillin G procaine suspension) is commercially available from Wyeth-Ayerst and is (2S,5R,6R)-3,3-Dimethyl-7-oxo-6-(2-phenylacetamido)-4-thia-1-azabicyclo [3.2.0]heptane-2-carboxylic acid compound with N,N′-dibenzylethylenediamine (2:1), tetrahydrate (Penicillin G benzathine) and (2S,5R,6R)-3,3-Dimethyl-7-oxo-6-(2-phenylacetamido)-4-thia-1-azabicyclo [3.2.0]heptane-2-carboxylic acid compound with 2-(diethylamino) ethyl p-amino benzoate compound (1:1) monohydrate (Penicillin G procaine).

Bicillin L-A (Penicillin G benzathine suspension) is commercially available from Wyeth-Ayerst and is (2S,5R,6R)-3,3-Dimethyl-7-oxo-6-(2-phenylacetamido)-4-thia-1-azabicyclo [3.2.0]heptane-2-carboxylic acid compound with N,N′-dibenzylethylenediamine (2:1), tetrahydrate.

Geocillin (carbencillin indanyl sodium) is commercially available from Pfizer and is 1-(5-Indanyl)-N-(2-carboxy-3-3-dimethyl-7-oxo-4-thia-1-azabicyclo [3.2.0]hept-6-yl)-2-phenylmalonamate monsodium salt.

Mezlin (sterile mezlocillin sodium) is commercially available from Bayer and is the monohydrate sodium salt of 6-{D-2[3 [(methyl-sulfonyl)-2-oxo-imidazolidine-1-carboxamido]-2-phenyl acetamido} penicillanic acid.

Omnipen (ampicillin) is commercially available from Wyeth-Ayerst and is (2S,5R,6R)-6-[(R)-2-Amino-2-phenylacetamido]-3,3-dimethyl-7-oxo-4-thia-1-aza-bicyclo [3.2.0]heptane-2-carboxylic acid.

Pen-Vee K (penicillin V potassium) is commercially available from Wyeth-Ayerst and is the potassium salt of the phenoxymethyl analog of penicillin G.

Pfizerpen (penicillin G potassium) is commercially available from Pfizer and is monopotassium 3,3-dimethyl-7-oxo-6-(2-phenylacetamido)-4-thia-1-azabicyclo (3.2.0) heptane-2-carboxylate.

Pipracil (piperacillin sodium) is commercially available from Lederle and is 4-thia-1-azabicyclo [3.2.0]heptane-2-carboxylic acid, 6-[[[[(4-ethyl-2-3-dioxo-1-piperazinyl) carbonyl] amino] phenylacetyl] amino]-3,3-dimethyl-7-oxo-, monosodium salt, [2S-[2α,5α,6β(S*)]].

Spectrobid (bacampicillin HCl) is commercially available from Pfizer and is 1′-ethoxycarbonyloxyethyl-6-(D-α aminophenylacetamide)-penicillate hydrochloride.

Ticar (ticarcillin disodium) is commercially available from SmithKline Beecham and is N-(2-carboxy-3,3-dimethyl-7-oxo-4-thia-1-azabicyclo [3.2.0]hept-6-yl)-3-thiophenemalonamic acid disodium salt.

Timentin (ticarcillin disodium and clavulanate potassium) is commercially available from SmithKline Beecham and is N-(2-carboxy-3,3-dimethyl-7-oxo-4-thia-1-azabicyclo [3.2.0]hept-6-yl)-3-thiophenemalonamic acid disodium salt (ticarcillin disodium) and potassium (Z)-(2R,5R)-3-(2-hydroxyethylidene)-7-oxo-4-oxa-1-azabicyclo [3.2.0]heptane-2-carboxylate (clavulanate potassium).

Unasyn (ampicillin sodium/sulbactam sodium) is commercially available from Pfizer and is monosodium (2S,5R,6R)-6-[(R)-2-Amino-2-phenyl acetamido]-3,3-dimethyl-7-oxo-4-thia-1-aza-bicyclo [3.2.0]heptane-2-carboxylate (amipicillin sodium), and sodium penicillate sulfone; sodium (2S,5R)-3,3-dimethyl-7-oxo-4-thia-1-azabicyclo [3.2.0]heptane-2-carboxylate 4,4-dioxide (sulbactam sodium).

Zosyn (piperacillin sodium and tazobactam sodium) is commercially available from Lederle and is sodium (2S,5R,6R)-6[(R)-2-(4-ethyl-2,3-dioxo-1-piperazine-carboxamido)-2-phenylacetamido]-3,3-dimethyl-7-oxo-4-Thia-1-azabicylco-[3.2.0]heptane-2-carboxylate (piperacillin sodium), and sodium (2S,3S, 5R)-3-methyl-7-oxo-3-(1H-1,2,3-triazol-1-ylmethyl)-4-thia-1-azabicyclo [3.2.0]heptane-2-carboxylate-4,4-dioxide (tazobactam sodium).

Dicloxacillin Sodium is monosodium (2S,5R,6R)-6-(3-(2,6-dichlorophenyl)5-methyl-4-isoxazolecarboxamido]-3,3-dimethyl-7-OXO-4-thia-1-azabicyclo[3.2.0]heptane-2-carboxylate monohydrate.

Achromycin V (tetracycline HCl) is commercially available from Lederle and is the monohydrochloride of [4S-(4α,4aα,5aα,6β, 12aα,)]-4-(Dimethylamino)-1,4,4a,5,5a,6,11,12a-octahydro-3,6,10,12,12a-pentahydroxy-6-methyl-1,11-dioxo-2-naphthacenecarboxamide.

Declomycin (demeclocycline HCl) is commercially available from Lederle Laboratories and is 7-chloro-4-dimethylamino)-1,4,4a,5,5a,6,11,12a-octahydro-3,6,10,12,12a-pentahydroxy-1,11-dioxo-2-naphthacenecarboxamide monohydrochloride.

Dynacin (minocylcine HCl) is commercially available from Medicis and is [4S-(4α,4aα,5aα,12aα)]-4,7-bis (dimethylamino)-1,4,4a,5,5a,6,11,12a-octahydro-3,10,12,12a-tetrahydroxy-1,11-dioxo-2-napthacene carboxamide monochloride.

Minocin (minocycline hydrochloride) is commercially available from Lederle Laboratories and is [4S-(4α,4aα,5aα,12aα)]-4,7-bis (dimethylamino)-1,4,4a,5,5a,6,11,12a-octahydro-3,10,12,12a-tetrahydroxy-1,11-dioxo-2-napthacene carboxamide monochloride.

Monodox (Doxycycline monohydrate capsules) is commercially available from Oclassen and is α-6-deoxy-5-oxytetracycline.

Terramycin (oxytetracyline) is commercially available from Pfizer.

Vectrin (minocycline hydrochloride) is commercially available from Warner Chilcott Professional Products and is [4S-(4α,4aα,5aα,12ax)]-4,7-bis (dimethylamino)-1,4,4a,5,5a,6,11,12a-octahydro-3,10,12,12a-tetrahydroxy-1,11-dioxo-2-napthacene carboxamide monochloride.

Vibramycin Calcium (doxycycline sodium) is commercially available from Pfizer and is 4-(Dimethylamino)-1,4,4a,5,5a,6,11,12a-octahydro-3,5,10,12,12a-pentahydroxy-6-methyl-1,11-dioxo-2-napthacene-carboxamide monohydrate.

Vibramycin Hyclate (doxycycline hyclate) is commercially available from Pfizer and is α-6-deoxy-5-oxytetracycline.

Vibramycin Monohydrate (doxycycline monohydrate) is commercially available from Pfizer and is 4-(Dimethylamino)-1,4,4a,5,5a,6,11,12a-octahydro-3,5,10,12,12a-pentahydroxy-6-methyl-1,11-dioxo-2-napthacene-carboxamide monohydrate.

Vibra-Tabs (doxycycline hydrate) is commercially available from Pfizer and is α-6-deoxy-5-oxytetracycline.

Vibramycin (doxycycline) is commercially available from Pfizer and is 4-(Dimethylamino)-1,4,4a,5,5a,6,11,12a-octahydro-3,5,10,12,12a-pentahydroxy-6-methyl-1,11-dioxo-2-napthacene-carboxamide monohydrate.

Lincomycins is monosodium (2S,5R,6R)-6-(3-(2,6-dichlorophenyl)5-methyl-4-isoxazolecarboxamido]-3,3-dimethyl-7-OXO-4-thia-1-azabicyclo[3.2.0]heptane-2-carboxylate monohydrate.

Cleocin HCl (clindamycin HCl) is commercially available from Pharmacia & Upjohn and is Methyl 7-chloro-6,7,8-trideoxy-6-(1-methyl-trans-4-propyl-L-2-pyrrolidine carboxamido)-1-thio-L-threo-α-D-galacto-octopyranoside monohydrochloride.

Abelcet (amphotericin B lipid complex) is commercially available from Libosome Company, Inc. and is [1R-(1R*,3S*,5R*,6R*,9R*,11R*,15S*,16R*,17R*,18S*,19E,21E,23E,25E,27E,29E,31E,33R*,35S*,36R*,37S*)]-33-[(3-amino-3,6-dideoxy-β-D-mannopyranosyl)-oxy]-1,3,5,6,9,11,17,37-octahydroxy-15,16,18-trimethyl-13-oxo-14,39-dioxabicyclo [33.3.1] nonatriaconta-19,21,23,25,27,29,31-heptaene-36-carboxylic acid.

AmBisome (amphotericin B) is commercially available from Fujisawa Healthcare and is [1R-(1R*,3S*,5R*,6R*,9R*,11R*,15S*,16R*,17R*,18S*,19E,21E,23E,25E,27E,29E,31E,33R*,35S*,36R*,37S*)]-33-[(3-amino-3,6-dideoxy-β-D-mannopyranosyl)-oxy]-1,3,5,6,9,11,17,37-octahydroxy-15,16,18-trimethyl-13-oxo-14,39-dioxabicyclo [33.3.1] nonatriaconta-19,21,23,25,27,29,31-heptaene-36-carboxylic acid.

Amphotec (amphotericin B cholesterol sulfate complex) is commercially available from Sequus Pharmaceuticals, Inc. and is [1R-(1R*,3S*,5R*,6R*,9R*,11R*,15S*,16R*,17R*,18S*,19E,21E,23E,25E,27E,29E,31E,33R*, ³⁵S*,36R*,37S*)]-33-[(3-amino-3,6-dideoxy-β-D-mannopyranosyl)-oxy]-1,3,5,6,9,11,17,37-octahydroxy-15,16,18-trimethyl-13-oxo-14,39-dioxabicyclo [33.3.1] nonatriaconta-19,21,23,25,27,29,31-heptaene-36-carboxylic acid.

Ancobon (flucytosine) is commercially available from ICN Pharmaceuticals and is 5-fluorocytosine.

Diflucan (fluconazole) is commercially available from Pfizer Inc. and is 2,4-difluoro-α-α′-bis (1H-1,2,4-triazol-1-ylmethyl) benzyl alcohol.

Fulvicin P/G (ultramicrosize griseofulvin) is commercially available from Schering.

Fulvicin P/G 165 and 330 (ultramicrosize griseofulvin) is commercially available from Schering.

Grifulvin V (griseofulvin) is commercially available from Ortho Dermatological.

Gris-PEG (griseofulvin ultramicrosize) is commercially available from Allergan.

Lamisil (terbinafine hydrochloride) is commercially available from Novartis and is (E)-N-(6,6-dimethyl-2-hepten-4-ynyl)-N-methyl-1-naphthalenemethanamine hydrochloride.

Nizoral (ketoconazole) is commercially available from Janssen and is cis 1-acetyl-4-[4-[[2-(2,4-di-chlorophenyl)-2-(1H-imidazol-1-ylmethyl)-1,3-dioxolan-4-yl] methoxy] phenyl] piperazine.

Amphotericin B is [1R-(1R*,3S*,5R*,6R*,9R*,11R*,15S*,16R*,17R*,18S*,19E,21E,23E,25E,27E,29E,31E,33R*,35S*,36R*,37S*)]-33-[(3-amino-3,6-dideoxy-β-D-mannopyranosyl)-oxy]-1,3,5,6,9,11,17,37-octahydroxy-15,16,18-trimethyl-13-oxo-14,39-dioxabicyclo [33.3.1] nonatriaconta-19,21,23,25,27,29,31-heptacne-36-carboxylic acid.

Lotrimin (clotrimazole) is commercially available from Schering and is 1-(O-Chloro-α,α-diphenyl benzyl)imidazole.

Dapsone tablets (dapsone) is commercially available from Jacobus and is 4,4′-diaminodiphenyl-sulfone (DDS).

Diflucan (fluconazole) is commercially available from Pfizer and is 2,4-difluoro-α-α′-bis (1H-1,2,4-triazol-1-ylmethyl) benzyl alcohol.

Monistat-Derm cream (miconazole) is commercially available from Ortho Dermatological and is 1-[2,4-dichloro-β-{(2,4-dichlorobenzyl) oxy} phenethyl] imidazole mononitrate.

Mycostatin Cream (nystatin) is commercially available from Westwood-Squibb.

Sporanox (itraconazole) is commercially available from Janssen Pharmaceutical and is (±)-1-[(R*)-sec-butyl]-4-[p-[[2R*,4S*)-2-(2,4-dichlorophenyl)-2-(1H-1,2,4-triazol-1-ylmethyl)-1,3-dioxolan-4-yl] methoxy] phenyl]-1-piperazinyl] phenyl]-Δ2-1,2,4, triazolin-5-one mixture with (±)-1-[(R*)-sec-butyl]-4-[p-[[2R*,4S*)-2-(2,4-dichlorophenyl)-2-(1H-1,2,4-triazol-1-ylmethyl)-1,3-dioxolan-4-yl] methoxy] phenyl]-1-piperazinyl] phenyl]-Δ2-1,2,4, triazolin-5-one or (±)-1-[(RS)-sec-butyl]-4-[p-[4-[p-[[(2R,4S)-2-(2,4-dichlorophenyl)-2-(1H-1,2,3,4-triazol-1-ylmethy) 1,3-dioxolan-4-yl] methoxy] phenyl]-1-piperazinyl] phenyl]-Δ2-1,2,4, triazolin-5-one.

Aralen hydrochloride (chloroquine HCl) is commercially available from Sanofi Pharmaceuticals and is 7-(chloro-4-[[4-diethylamino)-1-methyl butyl] amino]-quinoline dihydrochloride.

Aralen phosphate (chloroquine phosphate) is commercially available from Sanofi Pharmaceuticals and is 7-(chloro-4-[[4-diethylamino)-1-methyl butyl] amino]-quinoline phosphate (1:2).

Daraprim (pyrimethamine) is commercially available from Glaxo Wellcome and is 5-(4-chlorophenyl)-6-ethyl-2,4-pyrimidinediamine.

Lariam (mefloquine HCl) is commercially available from Roche Laboratories and is (R*,S*)-(±)-α-2-piperidinyl-2,8-bis (trifluoromethyl)-4-quinoline methanol hydrochloride.

Plaquenil (hydroxychloroquine sulfate) is commercially available from Sanofi Pharmaceuticals and is 2-[[4-[7-chloro-4-quinolyl) amino] pentyl] ethylamino] ethanol sulfate (1:1).

Capastat sulfate (capreomycin sulfate) is commercially available from Dura Pharmaceuticals.

Myambutol (ethambutol hydrochloride) is commercially available from Lederle Laboratories.

Mycobutin (rifabutin capsules) is commercially available from Pharmacia & Upjohn and is 1′,4-didehydro-1-deoxy-1,4-dihydro-5′-(2-methylpropyl)-1-oxorifamycin XIV or (9S,12E,14S,15R,16S,17R,18R,19R,20S,21S,22E,24Z)-6,16,18,20-tetrahydroxy-1-1′-isobutyl-14-methoxy-7,9,15,17,19,21,25-heptamethyl-spiro [9,4-(epoxypentadeca [1,11,13] trienimino)-2H-furo [2′,3′:7,8] naphth [1,2-d]imidazole-2,4′-piperidine]-5,10,26-(3H,9H)-trione-16-acetate.

Nydrazid (isoniazid injection) is commercially available from Apothecon.

Paser (aminosalicylic acid) is commercially available from Jacobus and is 4-amino-2-hydroxy benzoic acid.

Priftin (rifapentine) is commercially available from Hoechst Marion Roussel and is rifamycin 3-[[(4-cyclo-pentyl-1-piperazinyl) imino] methyl] or 3[N-(4-cyclopentyl-1-piperazinyl)-formimyidoyl]-2,7-(epoxypentadeca [1,11,13] trienimino)naptho [2,1-b]furan-1,11 (2H)-dione 21-acetate.

Pyrazinamide tablets (pyrazinamide) is commercially available from Lederle Laboratories and is the pyrazine analogue of nicotinamide.

Rifadin (rifampin capsules) is commercially available from Hoechst Marion Roussel and is 3-[[(4-methyl-1-piperazinyl) imino] methyl] rifamycin or 5,6,9,17,19,21-hexahydroxy-23-methoxy-2,4,12,16,20,22-heptamethyl-8-[N-methyl-1-piperazinyl) formimidoyl]-2,7-(epoxy pentadeca [1,11,13] trienimino)naptho [2,1-b]furan-1,11 (2H)-dione 21-acetate.

Rifadin IV (rifampin for injection) is commercially available from Hoechst Marion Roussel and is 3-[[3-(4-methyl-1-piperazinyl) formimidoyl]-2,7-(epoxy pentadeca [1,1,13] trienimino) naphtho [2,1-b]furan-1,11 (2H)-dione 21-acetate.

Rifamate (rifampin and isoniazid) is commerically available from Hoechst Marion Roussel and is 3-(4-methyl-1-piperazinyliminomethyl) rifamycin SV (rifampin) and hydrazide of isonicotinic acid (isoniazid).

Rifater (rifampin, isoniazid and pyrazinamide) is commercially available from Hoechst Marion Roussel and is 3-(4-methyl-1-piperazinyliminomethyl) rifamycin SV (rifampin), hydrazide of isonicotinic acid (isoniazid), and pyrazine analogue of nicotinamide (pyrazinamide).

Seromycin (cycloserine capsules) is commercially available from Dura Pharmaceuticals and is 3-isoxazolidinone, 4-amino-, (R)-.

Streptomycin Sulfate is commercially available from Pfizer and is O-2-deoxy-2-(methylamino)-α-L-glyucopyransoyl-(1→2)-O-5-deoxy-3-C-formyl-α-L-lyxofuranosyl-(1→4)-N-N′-bis(aminoiminomethyl)-, sulfate (2:3) salt.

Tice BCG (BCG vaccine) is commercially available from Organon and is attenuated live Mycobacterium bovis strains Bacillus of Calmette and Guerin.

Cycloserine (seromycin capsules) is commercially available from Dura Pharmaceuticals and is 3-isoxazolidinone, 4-amino-, (R)-.

Nydrazid (Isoniazid) is commercially available from Apothecon and is the hydrazide of isonicotinic acid.

Urised (Methenamine) is commercially available from Poly Medica.

Trecator-SC (ethionamide tablets) is commercially available from Wyeth-Ayerst and is 2-ethylthioisonicotinamide.

Alferon N (interferon alfa-n3) is commercially available from Interferon Sciences and is interferon alfa-n3 (human leukocyte derived).

Crixivan (indinavir sulfate) is commercially available from Merck & Co., Inc. and is [1(1S,2R), 5(S)]-2,3,5-trideoxy-N-(2,3-dihydro-2-hydroxy-1H-inden-1-yl)-5-[2-[[1,1-dimethylethyl) amino] carbonyl]-4-(3-pyridinyl-methyl)-1-piperazinyl]-2-(phenylmethyl)-D-erythropentonamide sulfate (1:1).

Cytovene (ganciclovir) is commercially available from Roche and is 9-[[2-hydroxy-1 (hydroxymethyl) ethoxy] methyl] guanine.

Cytovene-IV (ganciclovir sodium) is commercially available from Roche and is 9-[[2-hydroxy-1 (hydroxymethyl) ethoxy] methyl] guanine.

Epivir (lamivudine) is commercially available from Glaxo Wellcome and is (2R,cis)-4-amino-1-(2-hydroxymethyl-1,3-oxathiolan-5-yl)-1H)-pyrimidin-2-one.

Famvir (famciclovir) is commercially available from SmithKline Beecham and is 2-[2-(2-amino-9H-purin-9-yl) ethyl]-1,3-propanediol diacetate.

Flumadine (rimantadine HCl) is commercially available from Forest and is alpha-methyltricyclo-[3.3.1.1/3.7] decane-1-methanamine hydrochloride.

Foscavir (foscamet sodium) is commercially available from Astra and is phosphonoformic acid, trisodium salt.

Hivid (zalcitabine) is commercially available from Roche and is 4-amino-1-beta-D-2′,3′, dideoxyribofuranosyl-2-(1H)-pyrimidone or 2′,3′-dideoxycytidine.

Intron A (interferon alfa-2b) is commercially available from Schering.

Invirase (saquinavir mesylate) is commercially available from Roche Labs and is N-tert-butyl-decahydro-2-[2(R)-hydroxy-4-phenyl-3(S)-[[N-(2-quinolylcarbonyl)-L-asparaginyl] amino] butyl-(4aS,8aS)-isoquinoline-3(S)-carboxamide methanesulfonate.

Norvir (ritonavir) is commercially available from Abbott and is 10-Hydroxy-2-methyl-5-(1-methylethyl)-1-[2-(1-methylethyl)-4-thiazolyl]-3,6-dioxo-8,11-bis(phenylmethyl)-2,4,7,12-tetraazatridencan-B-oic acid, 5-thiazolyl methyl ester [5S-(5R*,8R*,10R*,11R*)].

Rebetron combination therapy, which contains Rebetrol (ribavirin which is 1-β-D-ribofuranosyl-1H-1,2,4-triazole-3-carboxamide) and Intron A (inteferon alfa-2b), is commercially available from Schering.

Rescriptor (delavirdine mesylate) is commercially available from Pharmacia & Upjohn and is pieperazine, 1-[3-[(1-methylethyl) amino]-2-pyridinyl]-4-[[5(methylsulfonyl)-amino]-1H-indol-2-yl] carbonyl], monomethanesulfonate.

Retrovir (ziduvudine) is commerically available from Glaxo Wellcome and is 3′-azido-3′-deoxythymidine.

Retrovir IV (zidovudine) is commercially available from Glaxo-Wellcome and is 3′-azido-3′-deoxythymidine.

Symmetrel (amantadine hydrochloride) is commercially available from Endo Pharmaceuticals and is 1-adamantanamine hydrochloride.

Synagis (palivizumab) is commercially available from MedImmune Inc. and is humanized monoclonal antibody (IgG1 κ).

Valtrex (valacyclovir HCl) is commercially available from Glaxo Wellcome and is L-valine, 2-[(2-amino-1,6-dihydro-6-oxo-9H-purin-9-yl) methoxy] ethyl ester, monohydrochloride.

Videx (didanosine) is commercially available from Bristol-Myers Squibb Oncology/Immunology and is 2′,3′-di-deoxyinosine.

Viracept (nelfinavir mesylate) is commercially available from Agouron and is [3S-[2(2S*,3S*), 3α,4aβ,8aβ]]-N-(1,1-dimethylethyl)decahydro-2-[2-hydroxy-3-[(3-hydroxy-2-methylbenzoyl)amino]-4-(phenylthio)butyl]-3-isoquinolinecarboxcamide mono-methanesulfonate (salt).

Viramune (nevirapine) is commercially available from Roxane and is 11-cyclopropyl-5,11-dihydro-4-methyl-6H-dipyrido [3,2-b:2′,3′-] [1,4] diazepin-6-one.

Virazole (ribavirin) is commercially available from ICN and is 1-beta-D-ribofuranosyl-1H-1,2,4-triazole-3-carboxamide.

Vistide (cidofovir) is commercially available from Gilead Sciences and is 1-[(S)-3-hydroxy-2-(phosphonomethoxy)propyl]cytosine dihydrate (HPMPC).

Zerit (stavudine (d4T)) is commercially available from Bristol-Myers Squibb Oncology/Immunology and is 2′,3′-didehydro-3′deoxythymidine.

Symmetrel Syrup (amantadine HCl) is commercially available from Endo Labs and is 1-adamantanamine hydrochloride.

Combivir Tablets (lamiduvine) is commercially available from Glaxo Wellcome and is 2′,3′-didehydro-3′-deoxythymidine.

Zovirax (acyclovir) is commercially available from Glaxo Wellcome and is 2-amino-1,9-dehydro-9-[(2-hydroxyethyoxy)methyl]-6H-purin-6-one.

Dapsone Tablets (dapsone) is commercially available from Jacobus and is 4,4′-diaminodiphenylsulfone (DDS).

Daraprim (pyrimethamine) is commercially available from Glaxo Wellcome and is 5-(4-chlorophenyl)-6-ethyl-2,4-pyrimidinediamine.

Flagyl 375 (metronidazole) is commercially available from Searle and is 2-Methyl-5-nitro-imidazole-1-ethanol.

Flagyl ER Tablets (metronidazole) is commercially available from Searle and is 2-Methyl-5-nitro-imidazole-1-ethanol.

Flagyl I.V. (metronidazole) is commercially available from SCS and is 2-Methyl-5-nitro-imidazole-1-ethanol.

Furoxone (furazolidone) is commercially available from Roberts and is 3-(5-nitrofurfuryliden-amino)-2-oxazolidinone.

Mepron (atovaquone) is commercially available from Glaxo Wellcome and is trans-2-[4-(4-chlorophenyl)cyclohexyl]-3-hydroxy-1,4-naphthalenedione.

Neutrexin (trimetrexate glucuronate) is commercially available from U.S. Bioscience and is 2,4-diamino-5-methyl-6-[(3,4,5-trimethoxyanilino)methyl] quinazoline mono-D-glucuronate.

Cipro (ciprofloxacin HCl) is commercially available from Bayer and is the monohydrochloride monohydrate salt of 1-cyclopropyl-6-fluoro-1,4-dihydro-4-oxo-7-(1-piperazinyl)-3-quinolinecarboxylic acid.

Floxin (ofloxacin) is commercially available from Ortho-McNeil Pharmaceutical and is (±)-9-fluoro-2,3-dihydro-3-methyl-10-(4-methyl-1-piperazinyl)-7-oxo-7H-pyrido[1,3,3-de]-1,4-benzoxazine-6-carboxylic acid.

Levaquin (levofloxacin) is commercially available from Ortho-McNeil Pharmaceutical) and is (−)-(S)-9-fluoro-2,3-dihydro-3-methyl-10-(4-methyl-1-piperazinyl)-7-oxo-7H-pyrido[1,2,3-de]-1,4-benzoxazine-6-carboxylic acid hemihydrate.

Mazaquin (lomefloxacin HCl) is commercially available from Unimed and is monohydrochloride salt of (±)-1-ethyl-6,8-difluoro-1,4-dihydro-7-(3-methyl-1-piperazinyl)-4-oxo-3-quinolinecarboxylic acid.

Noroxin (norfloxacin) is commercially available from Merck and is 1-ethyl-6-fluoro-1,4-dihydro-4-oxo-7-(1-piperazinyl)-3-quinolinecarboxylic acid.

Penetrex (enoxacin) is commercially available from Rhone-Poulenc Rorer and is 1-ethyl-6-fluoro-1,4-dihydro-4-oxo-7-(1-piperazinyl)-1,8-naphthyridine-3-carboxylic acid sesquihydrate.

Raxar (grepafloxacin HCl) is commercially available from Glaxo Wellcome and is (±)-1-cyclopropyl-6-fluoro-1,4-dihydro-5-methyl-7-(3-methyl-1-piperazinyl)-4-oxo-3-quinolinecarboxylic acid monochloride sesquihydrate.

Trovan (trovafloxacin mesylate) is commercially available from Pfizer and is (1α,5α,6a)-7-(6-amino-3-azabicyclo[3.1.0]hex-3-yl)-1-(2,4-difluorophenyl)-6-fluoro-1,4-dihydro-4-oxo-1,8-naphthyridine-3-carboxylic acid, monomethanesulfonate.

Zagam (sparfloxacin) is commercially available from Rhöne-Poulenc Rorer and is 5-Amino-1-cyclopropyl-7-cis-3,5-dimethyl-1-piperazinyl)-6,8-difluoro-1,4, dihydro-4-oxo-3-quinolinecarboxylic acid.

Bactrim (trimethoprim and sulfamethoxazole) is commercially available from Roche Labs and is 2,4-diamino-5-(3,4,5-trimethoxybenzyl) pyrimidine (trimethoprim) and N1,-(5-methyl-3-isoxazolyl)sulfanilamide (sulfamethoxazole).

Bactrim DS (trimethoprim and sulfamethoxazole double strength) is commercially available from Roche Labs and is 2,4-diamino-5-(3,4,5-trimethoxybenzyl) pyrimidine (trimethoprim) and N1,-(5-methyl-3-isoxazolyl)sulfanilamide (sulfamethoxazole).

Pediazole (erythromicin ethylsuccinate and sulfisaxazole acetyl) is commercially available from Ross and is erythromicin 2′-(ethyl succinate) and N′ acetyl sulfisoxazole (sulfisoxizole is N-(3,4-Dimethyl-5-isoxazolyl)-N-sulfanilyl acetamide.

Septra (trimethoprim and sulfamethoxazole) is commercially available from Monarch and is 5-[(3,4,5-trimethoxyphenyl)methyl]-2,4-pyrimidinediamine (trimethoprim) and 4-amino-N-(5-methyl-3-isoxazolyl)benzenesulfonamide (sulfamethoxazole).

Septra DS (trimethoprim and sulfamethoxazole) is commercially available from Monarch and is 5-[(3,4,5-trimethoxyphenyl)methyl]-2,4-pyrimidinediamine (trimethoprim) and 4-amino-N-(5-methyl-3-isoxazolyl)benzenesulfonamide (sulfamethoxazole).

Co-trimoxazole is a combined chemotherapeutic agent consisting of trimethoprim (T) and the sulphonamide sulphamethoxazole (S); their ratio is 1:5. It is bactericidal by virtue of a sequential blockade of the folic acid synthesis in micoorganisms. The antimicrobial spectrum of co-trimoxazole includes many Gram-positive and Gram-negative aerobes, Chlamydias, nocardias, protozoas (pneumocystis carinii), etc. In addition to its use for pneumocystis, co-trimoxazole mainly has practical importance against Gram-positive aerobes (urinary tract infections), pneumococci, and haemophilus influenzae (respiratory tract infections and otitis). http://www.infomed.org/100drugs/ctrifram.html.

Bactrim I.V. Infusion (sulfamethoxazole) is commercially available from Roche Labs.

Pediazole (erythromicin ethylsuccinate and sulfisoxazole acetyl) is commercially available from Ross and is erythromicin 2′-(ethyl succinate) and N′ acetyl sulfisoxazole (sulfisoxizole is N-(3,4-Dimethyl-5-isoxazolyl)-N-sulfanilyl acetamide.

Furadantin (nitrofurantoin) is commercially available from Dura and is 1-[[(5-nitro-2-furanyl)methylene]amino]-2,4-imidazolidinedione.

Macrobid (nitrofurantoin monohydrate macrocrystals) is commercially available from Procter & Gamble Pharmaceuticals and is 1-[[[5-nitro-2-furanyl]methylene] amino]-2-4-imidazolidinedione monohydrate.

Macrodantin (nitrofurantoin macrocrystals) is commercially available from Procter & Gamble Pharmaceuticals and is 1-[[[5-nitro-2-furanyl]methylene] amino]-2-4-imidazolidinedione.

Monurol Sachet (fosfomycin tromethamine) is commercially available from Forest and is (1R,2S)-(1,2-epoxypropyl) phosphonic acid, compound with 2-amino-2-(hydroxymethyl)-1,3-propanediol (1:1).

NegGram Caplets (nalidixic acid) is commercially available from Sanofi and is 1-ethyl-1,4-dihydro-7-methyl-4-oxo-1,8-naphthyridine-3-carboxylic acid.

Septra (trimethoprim and sulfamethoxazole) is commercially available from Monarch and is 5-[(3,4,5-trimethoxyphenyl)methyl]-2,4-pyrimidinediamine (trimethoprim) and 4-amino-N-(5-methyl-3-isoxazolyl)benzenesulfonamide (sulfamethoxazole).

Septra DS (trimethoprim and sulfamethoxazole) is commercially available from Monarch and is 5-[(3,4,5-trimethoxyphenyl)methyl]-2,4-pyrimidinediamine (trimethoprim) and 4-amino-N-(5-methyl-3-isoxazolyl)benzenesulfonamide (sulfamethoxazole).

Urised (a combination of the antiseptics methenamine, methylene blue, phenyl salicylate, benzoic acid and parasympatholytics (atropine sulfate) hyoscyamine) is commercially available from Poly Medica.

Urobiotic-250 Capsules (oxytetracycline HCl, sulfamethizole and phenazopyridine HCl) is commercially available from Pfizer.

Uroqid Acid No. 2 Tablets (methenamine mandelate) is commercially available from Beach.

Bactroban (mupirocin) is commercially available from SmithKline Beecham and is (αE,2S,3R,4R,5S)-5-[(2S,3S,4S,5S)-2,3-Epoxy-5-hydroxy-4-methylhexyl]tetrahydro-3,4-dihydroxy-β-methyl-2H-pyran-2-crotonic acid, ester with 9-hydroxynonanoic acid, calcium salt (2:1), dihydrate.

Chloromycetin opthalmic (chloramphenical) is commercially available from Monarch and is (1) Acetamide, 2,2-dichloro-N-[2-hydroxy-1-(hydroxymethyl)-2-(4-nitrophenyl) ethyl]-, and (2) D-threo-(−)-2,2-Dichloro-N-[β-hydroxy-α-(hydroxymethyl)-p-nitrophenethyl] acetamide.

Cortisporin (neomycin and polymyxin f sulfates and hydrocortisone acetate cream) is commercially available from Monarch and is 21-(acetyloxy)-11β,17-dihydroxypregn-4-ene-3,20-dione.

Ilotycin (erythromycin opthalmic ointment) is commercially available from Dista and is (3R*,4S*,5S*,6R*,7R*,9R*,11R*,12R*,13S*,14R*)-4-[(2,6-dideoxy-3-C-methyl-3-O-methyl-α-L-ribo-hexopyranosyl) oxy]-14-ethyl-7,12,13-trihydroxy-3,5,7,9,11,13, hexamethyl-6-[[3,4,6-trideoxy-3-(dimethylamino)-β-D-xylo-hex-opyranosyl] oxy] oxacyclotetradecane-2,10-dione.

NeoDecadron (neomycin sulfate—dexamethasone sodium phosphate) is commercially available from Merck and is 9-fluoro-11β,17-dihydroxy-16α-methyl-21-(phosphonooxy)pregna-1,4-diene-3,20-dione disodium salt.

Polytrim (trimethoprim and polythyxin β sulfate opthalmic solution) is commercially available from Allergan and is 2,4-diamino-5-(3,4,5-trimethoxy]benzl)pyrimidine (trimethoprim) and the sulfate salt of polymyxin B1 and B2 (polythyxin β sulfate).

Terra-Cortril (oxytetracycline HCl and hydrocortisone acetate) is commercially available from Pfizer.

TobraDex (tobramycin and dexamethasone opthalmic suspension and ointment) is commercially available from Alcon and is O-3-Amino-3-deoxy-a-D-glucopyranosyl-(1→4)-O-[2,6-diamino-2,3,6-trideoxy-a-D-ribo-hexopyranosyl-1(1→6)]-2-deoxy-L-streptamine. Dexamethasone: Chemical Name: 9-Fluro-11b,17,21-trihydroxy-16a-methylpregna-1,4-diene-3,20-dione.

Vira-A opthalmic ointment, 3% (vidarabine) is commercially available from Monarch and is 9H-Purin-6-amine, 9-β-D-arabinofuranosyl-, monohydrate.

Chibroxin (norfloxacin opthalmic solution) is commercially available from Merck and is 1-ethyl-6-fluoro-1,4-dihydro-4-oxo-7-(1-piperazinyl)-3-quinolinecarboxylic acid.

Ciloxan opthalmic solution, (Ciprofloxacin HCl) is commercially available from Alcon and is the monohydro chloride monohydrate salt of 1-cyclopropyl-6-fluoro-1,4-dihydro-4-oxo-7-(1-piperazinyl)-3-quinoline-carboxylic acid.

Ciloxan opthalmic ointment, (Ciprofloxacin HCl) is commercially available from Alcon and is the monohydro chloride monohydrate salt of 1-cyclopropyl-6-fluoro-1,4-dihydro-4-oxo-7-(1-piperazinyl)-3-quinoline-carboxylic acid.

Ocuflox opthalmic solution (ofloxacin) is commercially available from Allergan and is (±)-9-Fluoro-2,3-dihydro-3-methyl-10-(4-methyl-1-piperazinyl)-7-oxo-7H-pyrido[1,2,3-de]-1,4 benzoxazine-6-carboxylic acid.

Blephamide opthalmic ointment (sulfacetamide sodium and prednisolone acetate) is commercially available from Allergan and is N-sulfanilyl-acetamide monosodium salt monohydrate (sulfacetamide sodium) and 11β,17,21-trihydroxypreyna-1,4-diene-3,20-dione 21-acetate (prednisolone acetate).

Blephamide opthalmic suspension (sulfacetamide sodium and prednisolone acetate) is commercially available from Allergan and is N-sulfanilyl-acetamide monosodium salt monohydrate (sulfacetamide sodium) and 11β,17,21-trihydroxypreyna-1,4-diene-3,20-dione 21-acetate (prednisolone acetate).

A/T/S (erythromycin) is commercially available from Hoescht Marion Roussel and is (3R*,4S*,5S*,6R*,7R*,9R*,11R*,12R*,13S*,14R*)-4-[(2,6-dideoxy-3-C-methyl-3-O-methyl-α-L-ribo-hexopyranosyl) oxy]-14-ethyl-7,12,13-trihydroxy-3,5,7,9,11,13, hexamethyl-6-[[3,4,6-trideoxy-3-(dimethylamino)-β-D-xylo-hex-opyranosyl] oxy] oxacyclotetradecane-2,10-dione.

Bactroban (mupirocin) is commercially available from SKB and is (αE,2S,3R,4R,5 S)-5-[(2S,3S,4S,5S)-2,3-Epoxy-5-hydroxy-4-methylhexyl]tetrahydro-3,4-dihydroxy-β-methyl-2H-pyran-2-crotonic acid, ester with 9-hydroxynonanoic acid, calcium salt (2:1), dihydrate.

Benzamycin (erythromycin-benzoyl peroxide topical gel) is commercially available from Dermik and is (3R*,4S*,5S*,6R*,7R*,9R*,11R*,12R*,13S*,14R*)-4-[(2,6-dideoxy-3-C-methyl-3-O-methyl-α-L-ribo-hexopyranosyl) oxy]-14-ethyl-7,12,13-trihydroxy-3,5,7,9,11,13, hexamethyl-6-[[3,4,6-trideoxy-3-(dimethylamino)-β-D-xylo-hex-opyranosyl] oxy] oxacyclotetradecane-2,10-dione (erythromycin).

Betadine (povidone-iodine) is commercially available from Purdue Frederick.

Cleocin T (clindamycin phosphate topical solution) is commercially available from Pharmacia & Upjohn and is L-threo-I-D-galacto-Octopyranoside, methyl 7-chloro-6,7,8-trideoxy-6-[[(1-methyl-4-propyl-2-pyrrolidinyl)-carbonyl] amino]-1-thio-, 2-(dihydrogen phosphate), (2S-trans)-.

Clindets (clindamycin phosphate pledgets) is commercially available from Stiefel and is methyl 7-chloro-6,7,8-trideoxy-6-(1-methyl-trans-4-propyl-L-2-pyrrolidinecarboxamido)-1-thio-L-threo-α-D-galacto-octopyranoside 2-(dihydrogen phospate).

Emgel (erythromycin) is commercially available from Glaxo Wellcome and is (3R*,4S*,5S*,6R*,7R*,9R*,11R*,12R*,13S*,14R*)-4-[(2,6-Dideoxy-3-C-methyl-3-O-methyl-α-L-ribo-hexopyranosyl)oxy]-14-ethyl-7,12,13-trihydroxy-3,5,7,9,11,13-hexamethyl-6-[[3,4,6-trideoxy-3-(dimethyl-amino)-β-D-xylo-hexopyranosyl]oxy]oxacyclotetradecane-2,10-dione.

Erycette (erythromycin topical solution) is commercially available from Ortho Dermatological and is (3R*,4S*,5S*,6R*,7R*,9R*,11R*,12R*,13S*,14R*)-4-[(2,6-Dideoxy-3-C-methyl-3-O-methyl-α-L-ribo-hexopyranosyl)oxy]-14-ethyl-7,12,13-trihydroxy-3,5,7,9,11,13-hexamethyl-6-[[3,4,6-trideoxy-3-(dimethyl-amino)-β-D-xylo-hexopyranosyl]oxy]oxacyclotetradecane-2,10-dione.

Klaron (sodium sulfacetamide lotion) is commercially available from Dermik.

Mycostatin (nystatin cream) is commercially available from Westwood-Squibb.

Theramycin Z (erythromycin topical solution) is commercially available from Medicis and is (3R*,4S*,5S*,6R*,7R*,9R*,11R*,12R*,13S*,14R*)-4-[(2,6-dideoxy-3-C-methyl-3-O-methyl-α-L-ribo-hexopyranosyl) oxy]-14-ethyl-7,12,13-trihydroxy-3,5,7,9,11,13, hexamethyl-6-[[3,4,6-trideoxy-3-(dimethylamino)-β-D-xylo-hex-opyranosyl] oxy] oxacyclotetradecane-2,10-dione.

T-Stat (erythromycin) is commercially available from Westwood-Squibb and is (3R*,4S*,5S*,6R*,7R*,9R*,11R*,12R*,13S*,14R*)-4-[(2,6-dideoxy-3-C-methyl-3-O-methyl-α-L-ribo-hexopyranosyl) oxy]-14-ethyl-7,12,13-trihydroxy-3,5,7,9,11,13, hexamethyl-6-[[3,4,6-trideoxy-3-(dimethylamino)-β-D-xylo-hex-opyranosyl] oxy] oxacyclotetradecane-2,10-dione.

Exelderm (sulconazole nitrate) is commercially available from Westwood-Squibb and is (±)-1-[2,4-dichloro-β-[(p-chlorobenzyl)-thio]-plenethyl] imidazole mononitrate;

Fungizone (amphotericin B oral suspension) is commercially available from Bristol-Myers Squibb and is [1R-(1R*,3S*,5R*,6R*,9R*,11R*, 15S*,16R*,17R*,18S*,19E,21E,23E,25E,27E,29E,31E,33R*,35S*,36R*,37S*)]-33-[(3-Amino-3,6-dideoxy-β-D-mannopyranosyl)-oxy]-1,3,5,6,9,11,17,37-octahydroxy-15,16,18-trimethyl-13-oxo-14,39-dioxabicyclo [33.3.1] nonatriaconta-19,21,23,25,27,29,31-heptaene-36-carboxylic acid.

Lamisil (terbinafine hydrochloride cream) is commercially available from Novartis and is (E)-N-(6,6-dimethyl-2-hepten-4-ynyl)-N-methyl-1-naphthalenemethanamine hydrochloride.

Loprox (ciclopiroxolamine) is commercially available from Hoescht Marion Roussel and is 6-cyclohexyl-1-hydroxy-4-methyl-2(1H)-pyridone, 2-amino-ethanol salt.

Lotrimin (clotrimazole) is commercially available from Schering and is 1-(O-Chloro-α,α-diphenyl benzyl)imidazole.

Lotrisone (clotrimazole and betamethasone diproprionate) is commercially available from Schering and is 1-(O-Chloro-α,α-diphenyl benzyl)imidazole (clotrimazole) and 9-Fluoro-11β,17,21-trihroxy-16β-methylpregna-1,4-diene-3,20-dione 17,21-diproprionate (betamethasone diproprionate).

Mentax (butenafine HCl) is commercially available from Penederm and is N-4-tert-butylbenzyl-N-methyl-1-naphthalenemethylamine hydrochloride.

Monistat-Derm (miconazole nitrate) is commercially available from Ortho Dermatological and is 1-[2,4-dichloro-β-{(2,4-dichlorobenzyl)oxy)}phenethyl]imidazole mononitrate.

Mycelex (clotrimazole) is commercially available from Alza and is [1-(o-chloro-α,α-diphenylbenzyl) imidazole.

Mycostatin (nystatin) is commercially available from Westwood-Squibb.

Naftin (naftifine HCl) is commercially available from Allergan and is (E)-N-Cinnamyl-N-methyl-1-naphthalene-methylamine hydrochloride.

Nizoral (ketoconazole) is commercially available from Janssen and is cis-1-acetyl-4[4-[[2-(2,4-dichorophenyl)-2-(1H-imidazol-1-ylmethyl)-1,3-dioxolan-4-yl]methoxy]phenyl]piperazine.

Nystop (nystatin) is commercially available from Paddock.

Oxistat (oxiconazole nitrate) is commercially available from Glaxo Wellcome and is 2′,4′-dichloro-2-imidazole-1-ylacetophenone (Z)-[O-(2,4-dichlorobenzyl)oxime], mononitrate.

Selsun Rx (2.5% selenium sulfide lotion) is commercially available from Ross.

Spectazole (econazole nitrate) is commercially available from Ortho Dermatological and is 1-[2-{(4-chorophenyl) methoxy}-2-(2,4-dichlorophenyl)-ethyl]-1H-imidazole mononitrate.

Denavir (penciclovir cream) is commercially available from SmithKline Beecham and is 9-[4-hydroxy-3-(hydroxymethyl) butyl]guanine.

Zovirax (acyclovir) is commercially available from Glaxo-Wellcome and is 2-amino-1,9-dihydro-9-(2-hydroxyethoxy)methyl-6H-purin-6-one.

Benzashave (benzoyl peroxide) is commercially available from Medicis.

Betadine (povidone-iodine) is commercially available from Purdue Frederick.

Betasept (chlorhexidine gluconate) is commercially available from Purdue Frederick.

Cetaphil (soap substitute) is commercially available from Galaderma.

Clorpactin WCS-90 (sodium oxychlorosene) is commercially available from Guardiam Laboratories.

Dapsone Tablets (dapsone) is commercially available from Jacobus and is 4,4′-diaminodiphenyl sulfone (DDS).

Desquam-E (benzoyl peroxide) is commercially available from Westwood-Squibb.

Desquam-X (benzoyl peroxide) is commercially available from Westwood-Squibb.

Hibiclens (chlorhexidine gluconate) is commercially available from Zeneca.

Hibistat (chlorhexidine gluconate) is commercially available from Zeneca.

Impregon (tetrachlorosalicylanilide 2%) is commercially available from Fleming.

MetroCream (metronidazole) is commercially available from Galaderma and is 2-methyl-5-nitro-1H-imidazole-1-ethanol.

MetroGel (metronidazole) is commercially available from Galaderma and is 2-methyl-5-nitro-1H-imidazole-1-ethanol.

Noritate (metronidazole) is commercially available from Dermik and is 2-methyl-5-nitro-1H-imidazole-1-ethanol.

pHisoHex (hexachlorophene detergent cleanser) is commercially available from Sanofi and is Phenol,2,2′-methylene-bis[3,4,6-trichloro-].

Sulfacet-R (sodium sulfacetamide 10% and sulfur 5%) is commercially available from Dermik.

Sulfamylon (matenide acetate) is commercially available from Bertek and is α-amino-p-toluenesulfonamide monoacetate.

Triaz (benzoyl peroxide) is commercially available from Medicis.

Vanoxide-HC (benzoyl peroxide hydrocortisone) is commercially available from Dermik and is 11β,17,21-trihydroxypregn-4-ene-3,20-dione (hydrocortisone).

Acticin (permethrin) is commercially available from Penederm and is (±)-3-phenoxybenzyl 3-(2,2-dichlorovinyl)-2,2-dimethylcyclopropanecarboxylate.

Elimite (permethrin) is commercially available from Allergan and is (±)-3-phenoxybenzyl 3-(2,2-dichlorovinyl)-2,2-dimethylcyclopropanecarboxylate.

Eurax (crotamiton) is commercially available from Westwood-Squibb and is N-ethyl-N-(o-methylphenyl)-2-butenamide.

Lindane Lotion USP 1% (lindane) is commercially available from Alpharma.

Efudex (fluorouracil) is commercially available from ICN and is 5-flouro-2,4 (1H,3H)-pyrimidinedione.

Fluoroplex (fluorouracil) is commercially available from Allergan and is 5-flouro-2,4 (1H,3H)-pyrimidinedione.

Furadantin Oral Suspension (nitrofurantoin) is commercially available from Dura and is 1-[[5-nitro-2-furanyl)methylene]amino]-2,4-imidazolidine dione.

Zyvox (linezolid) is commercially available from Pharmacia & Upjohn.

It is appreciated that those skilled in the art understand that the antibiotic useful in the present invention is the biologically active compound present in any of the antibiotic drugs disclosed above. For example, Azactam (aztreonam) is typically available as an injectable solution. The antibiotic, however, is (z)-2-[[[(2-amino-4-thiazolyl) [[(2S,-3S)-2-methyl-4-oxo-1-sulfo-3-azetidinyl] carbamoyl] methylene] amino] oxy]-2-methyl propionic acid. Physician's Desk Reference (PDR), Medical Economics Company (Montvale, N.J.), (53rd Ed.), pp. 820-823, 1999.

The composition can further include alpha carotene, beta carotene, vitamin A, or a combination thereof; in any suitable and effective amount.

Xanthophylls

As used herein, “xanthophylls” refers to any of several yellow accessory pigments which found in plant leaves, egg yolks and human blood plasma, these pigments are oxygenated derivatives of carotenes and are involved in photosynthesis, for example lutein, violaxanthin and neoxanthine. In one embodiment, the xanthophylls can be derived from alfalfa, clove, kale, spinach, squash, black bean tops, sea-weed, leafy green vegetable, or any combination thereof. Specifically, the xanthophylls can include lutein, zeaxanthin, or a combination thereof.

Any suitable xanthophylls can be employed provided:

(1) the xanthophylls has the desired therapeutic and/or prophylactic properties (e.g., the xanthophylls effectively treats wrinkles); and

(2) the xanthophylls remains stable in the composition. Preferably, the stability is over a prolonged period of time, e.g., up to about 3 years, up to about 1 year, or up to about 6 months, typically experienced in the manufacturing, packaging, shipping, and/or storage of the composition. The specific xanthophylls will preferably be non-toxic to mammals (e.g., humans) and will be suitable for medicinal use (e.g., topically and/or via inhalation). The specific xanthophylls will also preferably comply with any controlling or governing body of law, e.g., FDA regulations.

As used herein, “lutein” refers to a compound having the formula: 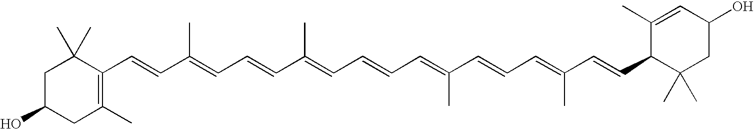
which is chemically designated as (3R,3′R,6′R)-β,ε-carotene-3,3′-diol. Lutein is a substance of a strongly marked yellow colour, extracted from the yolk of eggs, and from the tissue of the corpus luteum. Lutein is not made in the body and must be obtained from food or vitamin supplements. Lutein is found in large amounts in green, leafy vegetables such as spinach; and legumes such as alfalfa.

The esterified form of lutein refers to a compound having the formula: 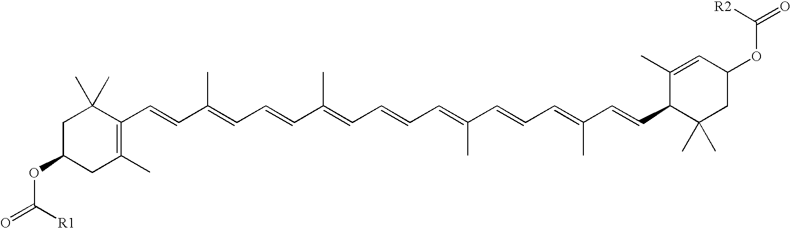
wherein R1 and R2 are radicals derived from fatty acids such as palmitic acid.

As used herein, “zeaxanthin” refers to a compound having the formula: 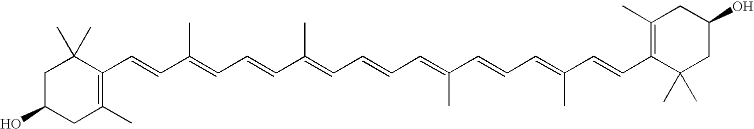
which is chemically designated as beta-carotene-3,3′-diol. Zeaxanthin is a carotene found in corn, fruits, seeds, and egg yolk.

The xanthophylls can be present in any appropriate and suitable amount, provided:

(1) the amount of xanthophylls has the desired therapeutic and/or prophylactic properties (e.g., the xanthophylls effectively kills or inactivates pathogens associated with acne); and

(2) the amount of xanthophylls remains stable in the composition. Preferably, the stability is over a prolonged period of time, e.g., up to about 3 years, up to about 1 year, or up to about 6 months, typically experienced in the manufacturing, packaging, shipping, and/or storage of the composition. The specific amount of xanthophylls will preferably be non-toxic to mammals (e.g., humans) and will be suitable for medicinal use (e.g., topically and/or via inhalation). The specific amount of xanthophylls will also preferably comply with any controlling or governing body of law, e.g., FDA regulations.

Typically, the amount of xanthophylls present in the composition will depend upon the specific compound or compounds employed as the xanthophylls. Specifically, the xanthophylls can be present in about 0.01 wt. % to about 99.9 wt. % of the composition. More specifically, the xanthophylls can be present up to about 50 wt. % of the composition, up to about 25 wt. % of the composition, up to about 20 wt. % of the composition, up to about 10 wt. % of the composition, or up to about 5 wt. % of the composition. In specific embodiments of the invention, the xanthophylls can be present in about 0.5 wt. % of the composition to about 25 wt. % of the composition, in about 1.0 wt. % of the composition to about 15 wt. % of the composition, or in about in about 1.0 wt. % of the composition to about 10 wt. % of the composition.

As used herein, “treating” or “treat” includes (i) preventing a pathologic condition (e.g., respiratory infection) from occurring (e.g. prophylaxis); (ii) inhibiting the pathologic condition (e.g., respiratory infection) or arresting its development; and (iii) relieving the pathologic condition (e.g., respiratory infection), or symptoms related to the pathologic condition.

As used herein, “mammal” refers to a class of vertebrate animals of more than 15,000 species, including humans, distinguished by self-regulating body temperature, hair, and in the females, milk-producing mammae. Specifically, mammal can refer to a human.

Humectant

The composition can optionally include one or more humectants to provide a moistening effect to the skin surface. Any suitable humectant can be employed, provided the humectant effectively provides a moistening effect to the skin surface, and the humectant remains stable in the composition. Preferably, the stability is over a prolonged period of time, e.g., up to about 3 years, up to about 1 year, or up to about 6 months, typically experienced in the manufacturing, packaging, shipping, and/or storage of the composition. One suitable humectant is glycerin. Other suitable humectants include polyhydric alcohols such as ethylene glycol, propylene glycol, triethylene glycol, tetraethylene glycol, sorbitol, and combinations thereof.

Any suitable amount of humectant can be employed, provided the amount of humectant effectively provides a moistening effect to the skin surface and the amount of humectant effectively remains stable in the composition. Preferably, the stability is over a prolonged period of time, e.g., up to about 3 years, up to about 1 year, or up to about 6 months, typically experienced in the manufacturing, packaging, shipping, and/or storage of the composition. Typically, the suitable amount of humectant will depend upon the specific humectant employed. For example, glycerin can be employed as the humectant, in about 25.0 wt. % to about 70.0 wt. % or in about 40.0 wt. % to about 55.0 wt. % of the composition.

Skin Protectant or Skin Conditioner

The composition can optionally include a skin protectant (i.e., topical moisturizer or skin conditioner). Any suitable skin protectant can be employed, provided the skin is effectively protected or moisturized and the skin protectant remains stable in the composition. Preferably, the stability is over a prolonged period of time, e.g., up to about 3 years, up to about 1 year, or up to about 6 months, typically experienced in the manufacturing, packaging, shipping, and/or storage of the composition. Additionally, it is preferable that the skin conditioner is medicinally acceptable for topical use in humans.

Suitable topical moisturizers include, e.g. calamine, aloe, lanolin, glycerin, Vitamin E, Vitamin E acetate, famesol, glycyrrhetinic acid, aluminum hydroxide gel, cocoa butter, propylene glycol, ethylene glycol, triethylene glycol, hard fat, kaolin, mineral oil, petrolatum, topical starch, white petroleum, cod liver oil, shark liver oil, zinc oxide, or any combination thereof. Additional suitable topical moisturizers are disclosed, e.g., in U.S. Pat. Nos. 6,096,334; 6,096,033; 5,741,510; 5,536,263; 4,675,009; 4,307,717; 4,274,420; 5,976,565; 5,536,263; and references cited therein.

As used herein, “aluminum hydroxide gel” refers to a suspension containing aluminum oxide (Al₂O₃), mainly in the form of a hydroxide. It is typically obtained by drying the product of interaction in aqueous solution of an aluminum salt with ammonium or sodium carbonate.

As used herein, “cocoa butter” refers to a fatty substance in cocoa beans; a thick oily solid obtained from cocoa beans and used in making chocolate, cosmetics, and suntan oil. Also known as threobroma oil, it lubricates and softens the skin.

As used herein, “topical starch” refers to cornstarch.

As used herein, “kaolin” refers to aluminum silicate; powdered and freed from gritty particles by elutriation. Kaolin refers to the name of the locality in China where the substance is found in abundance.

As used herein, “white petroleum” refers to a purified mixture of hydrocarbons obtained from petroleum. A bleached version of yellow soft paraffin, it is used as an emollient and as a base for ointments. It is odorless when rubbed into the skin and not readily absorbed.

As used herein, “mineral oil” refers to the heavy liquid petrolatum; liquid paraffin or petroleum; a mixture of liquid hydrocarbons obtained from petroleum, and is typically used as a vehicle in medicinal preparations.

As used herein, “petrolatum” refers to petroleum jelly; a yellow soft paraffin; a yellowish mixture of the softer members of the paraffin or methane series of hydrocarbons, obtained from petroleum as an intermediate product in the distillation; typically used as a soothing application to burns and abrasions of the skin, and as a base for ointments.

As used herein, “cod liver oil” refers to the partially destearinated fixed oil extracted from the fresh livers of Gadus morrhuae and other species of the family Gadidae, containing Vitamins A and D.

As used herein, “shark liver oil” refers to the oil extracted from the livers of sharks, mainly of the species Hypoprion brevirostris; a rich source of Vitamins A and D.

As used herein, “zinc oxide” refers to ZnO, which is typically used as a protective ointment.

As used herein, “calamine” is a pink powder of zinc oxide and a skin protectant containing about 98% zinc oxide and about 0.5% ferric oxide; “aloe” is the dried latex of leaves of Curaco Aloe (Aloe barbadenis Miller, Aloe vera Linne) or Cape Aloe (Aloe ferox Miller and hybrids), of the family Liliacaea. Aloe is commercially available as Aloe Vera Gel from Terry Laboratories (Melbourne, Fla.). Aloe Vera Gel is commercially available as Aloe Vera Gel 40X (20.0 wt. % solution in water), Aloe Vera Gel 1X (0.5 wt. % solution in water), Aloe Vera Gel 10X (5.0 wt. % solution in water), or solid Aloe Vera. The solid Aloe Vera can be dissolved in a carrier, such as water, to the desired concentration. In addition, the commercially available forms of Aloe Vera are optionally available as decolorized Aloe Vera.

As used herein, “Vitamin E” is 3,4-dihydro-2,5,7,8-tetramethyl-2-(4,8,12-trimethyltridecyl)-2H-1-benzopyran-6-ol; “Vitamin E acetate” is 3,4-dihydro-2,5,7,8-tetramethyl-2-(4,8,12-trimethyltridecyl)-2H-1-benzopyran-6-ol acetate; “lanolin” is the fat-like secretion of the sebaceous glands of sheep (i.e., complex mixture of esters and polyesters of 33 high molecular weight alcohols and 36 fatty acids) which is deposited onto the wool fibers; “famesol” is 3,7,11-trimethyl-2,6,10-dodecatrien-1-ol. Famesol is commercially available from American Radiolabeled Chemicals (ARC) (St. Louis, Mo.), and “glycyrrhetinic acid” is a pentacyclic triterpenoid derivative of the beta-amyrin type and is shown below:

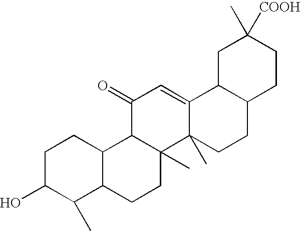

Any suitable amount of skin protectant can be employed, provided the suitable amount of skin protectant effectively protects or moisturizes the skin and the effective amount of skin protectant remains stable in the composition. Preferably, the stability is over a prolonged period of time, e.g., up to about 3 years, up to about 1 year, or up to about 6 months, typically experienced in the manufacturing, packaging, shipping, and/or storage of the composition. Additionally, it is preferable that the amount of skin conditioner employed is medicinally acceptable for topical use in humans.

Specifically, the skin protectant can be present up to about 20.0 wt. %, up to 10.0 wt. %, up to 5.0 wt. %, or up to 2.0 wt. % of the composition. The suitable and effective amount of skin protectant will depend in part upon the specific skin protectant present in the composition. For example, Aloe Vera Gel, 10X can be present up to about 20.0 wt. % of the composition, up to about 10.0 wt. % of the composition, up to about 5.0 wt. % of the composition, or up to about 1.0 wt. % of the composition. In addition, Vitamin E acetate can be present up to about 10.0 wt. % of the composition, up to about 5.0 wt. % of the composition, up to about 3.0 wt. % of the composition, up to about 2.0 wt. % of the composition, or up to about 1.0 wt. % of the composition. Preferably, the skin conditioner will be present in an amount that is consistent with any State or Federal regulations.

Preservative

The composition can optionally include a preservative that is useful for preventing bacterial growth, mold growth, fermentation, and/or decomposition. As used herein, “preservative” refers to any substance which prevents bacterial growth, mold growth, fermentation, and/or decomposition. Concise Chemical and Technical Dictionary, 4th enlarged edition, Chemical Publishing Co., Inc., NY, N.Y. p. 939 (1986). Any suitable preservative can be employed, provided the preservative effectively prevents bacterial growth, mold growth, fermentation, and/or decomposition; and the preservative remains stable in the composition. Preferably, the stability is over a prolonged period of time, e.g., up to about 2 years, up to about 1 year, or up to about 6 months, typically experienced in the manufacturing, packaging, shipping, and/or storage of the composition.

Suitable preservatives include, e.g., quat-15, parabens, dichlorobenzyl alcohol, ethylene diamine tetreacetic acid, formaldehyde, gum benzoin, imidazolidinyl urea, phenyl-mercuric acetate, poly aminopropyl biguanide, proply gallate, sorbic acid, cresol, chloroacetamide sodium benzoate, chloromethyl-methylisothiazolinone, chloromethyl-methylisothiazolon, chloromethyl-methylisothiazolinone benzalkonium chloride, an octylisothiazolinone benzimidazol-compound, chloromethyl-methylisothiazolinone octylisothiazolinone, o-phenylphenol benzisothiazolinone, o-phenylphenol benzisothiazolinone, benzisothiazolinone, an aliphatic amine of 2-thiopyridineoxide, benzoic acid, editic acid, phenolic acid, benzyl alcohol, isopropyl alcohol, benzenethonium chloride, bronopol, cetrimide, chlorohexidine, chlorobutanol, chlorocresol, phenol, phenoxyethanol, phenyl ethyl alcohol, phenylmercuric acetate, phenylmercuric borate, phenylmercuric nitrate, potassium sorbate, proplyene glycol, sodium benzoate, sodium propionate, thimerosol, and medicinally acceptable salts thereof. Preferably, the preservative is quat-15, which is commercially available from Dow Chemical (Midland Mich.); methyl paraben; ascorbic acid; or a combination thereof.

The preservative can be employed in any suitable amount provided the amount of preservative effectively prevents bacterial growth, mold growth, fermentation, and/or decomposition and the effective amount of preservative remains stable in the composition. Preferably, the stability is over a prolonged period of time, e.g., up to about 3 years, up to about 1 year, or up to about 6 months, typically experienced in the manufacturing, packaging, shipping, and/or storage of the composition. The preservative can be present up to about 99.9 wt. % of the composition, up to about 20.0 wt. % of the composition, up to 5.0 wt. % of the composition, or up to 1.5 wt. % of the composition. The amount of preservative present in the composition will typically depend upon the specific compound or compounds employed as the preservative. For example, quat-15 can be employed in about 0.01 wt. % to about 1.5 wt. % of the composition, in about 0.05 wt. % to about 0.15 wt. % of the composition, or in about 0.08 wt. % to about 0.12 wt. % of the composition.

Complexing Agent

In one embodiment of the present invention, the composition can include xanthophylls that is not soluble and/or stable either without a solvent or with the specific solvent, in the amount employed. The use of a complexing agent can be employed to modulate (i.e., regulate) the solubility, stability, and/or the volatility of the xanthophylls in the composition. Any suitable complexing agent can be employed, provided the complexing agent effectively solubilizes and/or stabilizes the xanthophylls and the complexing agent remains stable in the composition over a prolonged period of time. Preferably, the stability is over a prolonged period of time, e.g., up to about 3 years, up to about 1 year, or up to about 6 months, typically experienced in the manufacturing, packaging, shipping, and/or storage of the composition. In addition, any suitable amount of complexing agent can be employed, provided the amount of complexing agent effectively solubilizes and/or stabilizes the xanthophylls and the amount of complexing agent remains stable in the composition over a prolonged period of time.

Suitable specific complexing agents include, e.g., cyclodextrins. As used herein, a “cyclodextrin” refers to a non-reducing cyclic oligosaccharide with at least 6 anhydroglucose units linked by alpha 1,4 bonds to form a ring. Cyclodextrins are typically produced by the action of the enzyme cyclodextrin glucosyltransferase [CGT-ase] on starch. The most common cyclodextrins include alpha, beta, and gamma cyclodextrins, which have six, seven, or eight, respectively, anhydroglucose units in the ring structure. All of the hydroxyl groups in cyclodextrins are oriented to the outside of the ring while the glucosidic oxygen and two rings of the non-exchangeable hydrogen atoms are directed towards the interior of the cavity. This combination gives cyclodextrins a hydrophobic inner cavity and a hydrophilic exterior. See, e.g., the Cerestar website (http://www.cerestar.com); the Betadexcyclodextrin website (http://www.betadexcyclodextrin.com); and M. L. Bender and M. Komiyama, Cyclodextrin Chemistry, Springer, Berlin, 1978.

Cyclodextrins are enzymatically-modified starches formed by the action of the enzyme cyclodextrin glucosyltransferase on starch. They are doughnut-shaped molecules, which can interact with organic molecules to form complexes. It is also possible for some organic molecules and some inorganic salts to associate with the hydroxyl groups of the cyclodextrin. Three cyclodextrins are typically formed, alpha, beta, and gamma cyclodextrin, which contain six, seven, or eight glucose molecules in the ring, respectively. The electron-dense glycosidic oxygen atoms are oriented inward and line the cavity. The hydroxyl groups are directed toward the outside of the ring. These hydrophilic groups interact with the water to give the cyclodextrins their aqueous solubility properties. The hydrogen and glycosidic oxygen atoms lining the cavity give the cyclodextrin molecule its hydrophobic character and its ability to interact with organic molecules to form complexes. Because of the free rotation of the C-6 carbon, this end of the cyclodextrin cavity is narrower than the end with the C-2 and C-3 hydroxyls.

Derivatives of cyclodextrin can be obtained, e.g., by replacing one or more hydroxyl groups with a suitable radical (i.e., pendant group). Suitable pendant groups include, e.g., sulfinyl; sulfonyl; phosphate; (C₁-C₁₂)alkyl optionally substituted with one or more (e.g., 1, 2, 3, or 4) hydroxy, carboxy, carbonyl, acyl, oxy, oxo; or a combination thereof. Suitable specific pendant groups include methyl, ethyl, hydroxypropyl, carboxymethyl, sulfate, phosphate, and an acrylate. For example, the specific pendant group can include (C₁-C₁₂)alkoxy optionally substituted with one or more hydroxy.

Specific suitable derivatives of cyclodextrin include, e.g., alpha-cyclodextrin sulfate, beta-cyclodextrin sulfate, gamma-cyclodextrin sulfate, alpha-hydroxypropyl cyclodextrin, beta-hydroxypropyl cyclodextrin, gamma-hydroxypropyl cyclodextrin, alpha-cyclodextrin phosphate, beta-cyclodextrin phosphate, and gamma-cyclodextrin phosphate.

Cyclodextrins are starches that have been specially modified by the action of an enzyme to make a water-soluble ring-shaped molecule, capable of holding another, oil-like organic substance in its ‘cavity’. Because of this unique property, cyclodextrins can be used to carry all kinds of active ingredients (e.g., drugs, fragrances, flavors, and vitamins) in a wide variety of formulations. Increased stability, water solubility, and controlled release are among the many application benefits. Specifically, cyclodextrins have the benefit of encapsulating a substance, thereby providing protection for the substance. This results in increased shelf-life and a reduced loss of degradation or decomposition. Cyclodextrins are themselves soluble in water, and can greatly increase the solubility of highly water insoluble substances. In addition, cyclodextrins can be used to control the release of a substance.

Suitable cyclodextrins include alpha cyclodextrins, beta cyclodextrins, and gamma cyclodextrins. Specifically, the cyclodextrin can be hydroxylpropyl beta cyclodextrin, hydroxylproplyl alpha cyclodextrin, or a combination thereof. In addition, the cyclodextrin can optionally be branched.

Suitable cyclodextrins, and derivatives thereof, can be found, e.g., at U.S. Pat. No. 5,376,641; U.S. Pat. No. 5,229,370; U.S. Pat. No. 4,383,992; the Cerestar website (http://www.cerestar.com); the Betadexcyclodextrin website (http://www.betadexcyclodextrin.com); French et al., Archives in Biochem. and Biophysics, Volume III, (1965) 153-150; the carbomer website (http://www.carbomer.com) and references cited therein.

In one embodiment of the present invention, the composition can further include a penetration enhancer effective to improve the penetration of the xanthophylls into and through bodily tissue (e.g., skin), with respect to a composition lacking the penetration enhancer. The penetration enhancer may generally be any penetration enhancer. Suitable penetration enhancers include, e.g., diethylene glycol monoethyl ether (transcutol), dimethyl sulfoxide (DMSO), propyleneglycol, ionic surfactants, non-ionic surfactants, anionic surfactants, isopropyl myristate (IPM), calcipotriene, detergents, emollients, chelators (e.g., calcium chelators such as EDTA, EGTA), and combinations thereof. Additional Examples of enhancers include Loramide DEA, Ethoxydiglycol, NMP, Triacetin, Propylene Glycol, Benzyl Alcohol, Sodium Laureth Sulfate, Dimethyl Isosorbide, Isopropyl Myristate, Olive Squalane, Medium Chain Triglyceride Oil (MCT Oil), Menthol, Isopropyl Palmitate, Isopropyl Isostearate, Propylene Glycol Monostearate, Lecithin, Diisopropyl Adipate, Diethyl Sebacate, Oleic Acid, Ethyl Oleate, Urea, Glyceryl Oleate, Caprylic/Capric Triglyceride, Propylene Glycol Dicaprylate/Dicaprate, Laureth-4, Oleth-2, Oleth-20, Propylene Carbonate, Nonoxynol-9,2-n-nonyl-1,3-dioxolane, C₇ to C₁₄-hydrocarbyl substituted 1,3-dioxolane, 1,3-dioxane, or acetal, and Nonoxynol-15. Specifically, the skin absorption enhancer can include diethylene glycol monoethyl ether (transcutol). As used herein, “diethylene glycol monoethyl ether” or “transcutol” refers to 2-(2-ethoxyethoxy)ethanol [CAS NO. 001893].

The penetration enhancer can be present in the composition in any suitable and appropriate amount (e.g., between about 1 wt. % and about 10 wt. %).

In one embodiment of the present invention, the composition can further include a keratolytic agent. As used herein, “keratolytic agent” refers to a substance that causes desquamation (loosening) and debridement or sloughing of the surface cells of the epidermis. Any suitable keratolytic agent can be employed, preferably the keratolytic agent effectively causes desquamation (loosening) and debridement or sloughing of the surface cells of the epidermis. Preferably, the keratolytic agent is pharmaceutically acceptable for topical use on humans. Suitable keratolytic agents include, e.g., alcloxa, resorcinol, or a combination thereof. As used herein, “alcloxa” refers to A1-chlorhydroxy allontoinate; and “resorcinol” refers to m-dihydroxybenzene or 1,3-benzenediol.

Any suitable and effective amount of keratolytic agent can be employed, provided the amount of keratolytic agent effectively causes desquamation (loosening) and debridement or sloughing of the surface cells of the epidermis. The keratolytic agent can include an amount of alkaline material (e.g., potassium hydroxide (KOH), sodium hydroxide (NaOH), etc.), effective to cause desquamation (loosening) and debridement or sloughing of the surface cells of the epidermis. Alternatively, the desquamation (loosening) and debridement or sloughing of the surface cells of the epidermis can be achieved with the use, e.g., of mechanical stripping, tape, etc. Alternatively, the desquamation (loosening) and debridement or sloughing of the surface cells of the epidermis can be achieved with the use, e.g., of radiant energy such as ultrasound, heat, etc., or with the use of photodynamic therapy.

Topical Acne Drug

The composition can further include a topical acne drug, useful to treat acne. The topical acne drug can be present in any appropriate and suitable amount, provided the amount of topical acne drug is effective to treat and/or prevent acne or a pimple and the amount of topical acne drug remains stable in the composition over a prolonged period of time. Preferably, the stability is over a prolonged period of time, e.g., up to about 3 years, up to about 1 year, or up to about 6 months, typically experienced in the manufacturing, packaging, shipping, and/or storage of the composition. Typically, the topical acne drug can be present in about 0.01 wt. % to about 99.9 wt. % of the composition. Specifically, the amount of topical acne drug present in the composition can be up to about 5.0 wt. % of the composition, up to 4.0 wt. % of the composition, up to 3.0 wt. % of the composition, or in about 0.5 wt. % to about 2.0 wt. % of the composition. Preferably, the topical acne drug and amount thereof will comply with FDA regulations (e.g., 21 C.F.R. Chapter 1, Section 333, Subpart D—Topical Acne Drug Products, Apr. 1, 2000 Edition).

The amount of topical acne drug present in the composition will typically depend upon the specific compound or compounds employed as the topical acne drug. For example, salicylic acid can be present up to about 99.9 wt. % of the composition, up to about 10.0 wt. % of the composition, up to 2.0 wt. % of the composition, or up to about 2.0 wt. % of the composition.

Specifically, resorcinol can be present up to about 2 wt. % of the composition, in accordance with 21 CFR Ch.1, §§ 333.320(a) and 333.310(a). Specifically, resorcinol monoacetate can be present up to about 3 wt. % of the composition, in accordance with 21 CFR Ch.1, §§ 333.320(b) 333.310(b). Specifically, salicylic acid can be present in about 0.5 wt. % to about 2.0 wt. % of the composition, in accordance with 21 CFR Ch.1, § 333.310(c). Specifically, sulfur can be present in about 3.0 wt. % to about 10.0 wt. % of the composition, in accordance with 21 CFR Ch.1, § 333.310(d).

The composition can be applied to any suitable skin surface of the patient. For example, when the composition is used to treat topical skin disorders (e.g., wrinkles, acne, etc.), the composition can be applied to that surface of the skin that is afflicted with the topical skin disorder. In such an embodiment, the composition can locally deliver a cosmetically and/or therapeutically effective amount of xanthophylls.

As used herein, a “lotion” refers to a liquid, usually an aqueous medicinal preparation containing one or more insoluble substances and applied externally for skin disorders; “cream” refers to an emulsified medicinal or cosmetic preparation; a semisolid emulsion of either the oil-in-water or the water-in-oil type, ordinarily intended for topical use; “gel” refers to a colloid in a more solid form than a solution; a jelly-like material formed by the coagulation of a colloidal liquid; many gels have a fibrous matrix and fluid filled interstices: gels are viscoelastic rather than simply viscous and can resist some mechanical stress without deformation; and “ointment” refers to a salve or unguent for application to the skin, specifically a semisolid medicinal preparation usually having a base of fatty or greasy material; an ointment has an oil base whereas a cream is water-soluble. (Webster's II New College Dictionary, Houghton Mifflin Company, New York (2001); Merriam Webster's Medical Desk Dictionary, Merriam-Webster. Incorporated, Springfield, Mass. (1996))

Age Associated Structural Changes

Although many of the effects of the aging of the human skin are the result of underlying structural changes which build up over a period of years and can only be detected histologically prior to young adult life, these changes and effects begin to appear clinically in young adults, namely those between about 20 and 30 years of age, and are generally evident about middle age, namely between about 35 and 45 years of age, and become more and more evident and pronounced thereafter, especially in persons excessively exposed to sunlight. The more apparent effects of aging have already been referred to above; and each is associated with one or more underlying structural changes in the skin. For example, blotchiness or mottling (hyperpigmentation) is due to accumulation of melanin in the basal cells of the epidermis. This happens because the reproduction of the cells slows down greatly with aging, allowing them much longer time to receive melanin from the surrounding pigment-producing melanocytes. By stimulating the proliferation of basal cells, pigment retention is prevented.

In addition to obvious cosmetic improvements in the skin, there are a number of other changes which are more important though less apparent, including loss of sensory acuity, reduced wound healing, decreased blood flow and decrease in the thickness of the skin. Older people have less sensitivity to pain and a longer response time. Thus, pain due to irritation or injury is not felt as soon or to the same extent as in young people with the result that superficially minor but potentially serious injuries may be sustained without the individual being aware of the injury until serious damage has occurred.

The surface temperature of the skin in older people is lower than the skin temperature in younger people, so that they often feel cold. This is one reason why the elderly retire to the sun-belt. Anatomically there is a great loss of small blood vessels so that physiologically the blood flow through the skin is greatly reduced. The skin becomes paler and cooler. Furthermore, the decreased blood supply decreases the rate at which irritants and toxins are cleared from the skin. Dangerous build-up of toxic agents can result.

Still further, the skin of older people is more easily torn than that of younger people, since both the epidermis and dermis become thinner with age and the fibrous matrix becomes structurally inferior. As a result, there is less bulk to protect underlying organs and therefore more risk of serious injury. Moreover, when wounds or injuries are sustained, healing of the wounds is much slower in older people.

The underlying causes of the above gross skin effects may be understood more readily from the following discussion of the specific changes in the epidermis and dermis as aging progress

1. Epidermis

With increasing age and exposure of a human to sun and other environmental traumas, cells divide at a slower rate (decreased capacity to renew themselves). They show marked irregularities in size, shape and staining properties; orderliness (polarity) from below to above is lost. The thickness of the epidermis decreases (atrophy). The horny layer which includes the barrier against water loss and penetration of chemicals becomes abnormal due to the shedding (exfoliation) of cells in large groups or clusters instead of as individual cells, resulting in roughness, scaling and dryness. There is loss of the orderly transformation of living epithelial cells into cornified dead cells which are shed at the surface, that is, differentiation is impaired. Aberrant differentiation results in numerous foci of abnormal epithelial growths or tumors, the most frequent of which are actinic keratoses. After many years these can transform into frank skin cancers called basal cell and squamous cell cancers. Pigment producing cells (melanocytes) can also become altered, forming flat, dark growths (lentigo melanoma) which may progress to malignant melanoma. The cells which make up these permalignant growths are eliminated by topical application of compounds such as xanthophylls and retinoids.

2. Dermis

The cells which make up the fibers of the dermis become smaller and sparser with increasing age, usually in sundamaged facial skin. There is a great loss of collagen fibers resulting in looseness and easy stretchability of the skin; elastic fibers become abnormal so that the skin does not promptly snap back after being stretched. Since the fibrous components comprise more than 90% of the bulk of skin of which 95% is collagen, the degradation of these fibers, especially collagen, is mainly responsible for wrinkling, laxness and loss of elasticity.

Small blood vessels become thin walled, dilated and often ruptured. Vascular supply thereby becomes compromised.

Beneficial Effects of Xanthophylls and Retinoids in combination with xanthophylls in accordance with the present invention:

(a) Increased proliferative activity of epidermal cells. This results in thickening of the epidermis with correction of atrophy. Cell renewal is quickened so that cells divide at a rate typical of younger skin. Treatment with xanthophylls and Retinoids in combination with xanthophylls in accordance with the invention can double the thickness of the epidermis. The stimulation of cell growth also results in faster wound healing. Experiments have been performed wherein blisters have been raised and the roofs cut off of the skins of individuals of various ages. Healing takes place in 2 or 3 weeks in young people, but takes much longer in older persons. For example, application of the retinoid tretinoin, vitamin A acid or all-trans retinoic acid (alone or in combination with xanthophylls) before raising the blister halves the healing time;

(b) Correction of abnormalities of differentiation. Retinoids in combination with xanthophylls regulate and control the physiologic behavior of epithelial tissue, assuring its stability and integrity. They correct and normalize abnormalities of differentiation. In sundamaged skin, the numerous foci of abnormal growth and segments of atypical, abnormal epidermis are corrected, reversed or eliminated. Fewer growths appear and progression to cancer is halted. Normalizing of the epidermis results in a smoother, less dry and rough skin, since cells are not only produced more rapidly, but exfoliation occurs by individual cells rather than in clusters or scales, thus improving the topography of the skin. Moreover, hyperpigmentary blotches and splotches are reduced by Retinoids in combination with xanthophylls, eliminating the mottled appearance of sundamaged skin;

(c) The metabolism of fibroblasts is increased. Fibroblasts synthesize the fibers of the dermis; new collagen is laid down, strengthening the physical foundation of the skin. Fibroblasts also make the ground substance which exists between the fibers, allowing these to glide past each other. The ground substance, known as acid mucopolysaccharides, is also responsible for the turgor and bounce of the skin. Retinoids in combination with xanthophylls stimulate the formation of new acid mucopolysaccharides.

Accordingly, retinoids in combination with xanthophylls promote the formation of a more normal dermis. Because of this activity, they have been found to promote and accelerate the healing of wounds in compromised tissue, of which aged dermis is an example. Further, the production of a new collagen layer not only repairs damaged skin but results in the effacement and prevention of fine wrinkles and lines;

(d) Vascularity is increased. Retinoids in combination with xanthophylls stimulate blood flow and promote the formation of new vessels. Blood flow is greatly reduced in aged, sundamaged skin. A brisker blood supply improves the physiologic competence of the skin and imparts a livelier, glowing appearance. Patients often say their skin feels “more alive”.

Several of the prior art treatments using retinoic acid as referred to above have claimed there is an increase in the blood flow in the skin. However, the increased blood flow from such short term treatments could result simply from vasodilation caused by the irritating effects of high concentrations of the acid. In contrast, the low sub-irritating concentrations of retinoids according to the present invention do not cause significant vasodilation. Over the long term there occurs formation of many new small blood vessels, markedly increasing the functional blood supply to the skin. As a result, the skin can react more effectively to external sources of damage and can then mount a more normal inflammatory response to fight infection. The increased blood supply allows the skin to clear irritants and toxins more quickly.

Still further, treatment with retinoids in combination with xanthophylls according to the present invention can raise the surface temperature of the skin by about ½ degree centigrade due to greater flow of blood. The increased blood flow can increase acuity to pain and irritation, and the skin can become more reactive to chemical insults. For example, experiments with highly drying and irritating cosmetics, soaps, perfumes, etc. have shown that young people will experience severe irritation within 3 or 4 days whereas it may take 2 to 3 weeks for an older person to note the same irritation. The increased sensitivity of the skin treated with retinoids provides an early warning system to older people so that too much damage is not done before the pain or irritation is felt.

Retinoids have historically been defined narrowly as including simply vitamin A (retinol) and its derivatives such as vitamin A aldehyde (retinal), vitamin A acid (retinoic acid), including the so called natural retinoids. However, subsequent research has resulted in a much larger class of chemical compounds that are termed retinoids due to their biological similarity to vitamin A and its derivatives. Compounds useful as retiniods in the present invention include all natural and/or synthetic analogues of vitamin A or retinol-like compounds which possess the biological activity of vitamin A in the skin, such as the control of epithelial cell differentiation of keratinocytes in the epidermis and/or stimulation of fibroplasia or new collagen synthesis in the dermis among other effects. Accordingly, as used herein for purposes of the present invention, the term “retinoid” will be understood to include any of the foregoing compounds. Examples of suitable retinoids for use in the present invention are set forth in Table I, although it will be understood that the invention is not limited thereto.

TABLE 1

| Retinoid | Amount (wt. %) |
|---|---|
| Isotretinoin | 0-10 |
| 13-cis-retinoic acid | 0-10 |
| ACCUTANE | 0-10 |
| Etretinate | 0-10 |
| TEGISON | 0-10 |
| (all-E)-9-(4-methoxy-2,3,6- | 0-10 |
| trimethylphenyl)-3,7-dimethyl-2,4,6,8- | |
| nonatetraenoic acid ethyl ester | |
| Etretin | 0-10 |
| (all-E)-9-(4-methoxy-2,3,6-trimethylphenyl)- | 0-10 |
| 3,7-dimethyl-2,4,6,8- | |
| nonatetraenoic acid | |
| Motretinate | |
| N-ethyl-9-(4-methoxy-2,3,6-trimethyl- | 0-10 |
| phenyl)-3,7-dimethyl-2,4,6,8-mono- | |
| tetraenamide | |
| (E,E)-9-(2,6-dichloro-4-methoxy-3- | |
| methylphenyl)-3,7-dimethyl-2,4,6,8- | |
| nonatetraenoic acid ethyl ester | |
| 7,8-didehydroretinoic acid | 0-10 |
| (E,E)-4-[2-methyl-4-(2,6,6-trimethyl-1- | 0-10 |
| cyclohexane-1-yl)-1,3- | |
| butadiamyl]benzoic acid | |
| (E,E)-4-[4-methyl-6-(2,6,6-trimethyl-1- | 0-10 |
| cyclohexane-1-yl)-1,3,5- | |
| hexatrienyl]benzoic acid | |
| (all-E)-3,7-dimethyl-9-(3-thienyl)-2- | 0-10 |
| 4,6,8-nonatetraenoic acid | |
| (E,E,E)-3-methyl-7-(5,6,7,8-tetrahydro- | 0-10 |
| 5,5,8,8-tetramethyl-2-naphthalenyl)- | |
| 2,4,6-octatrienoic acid | |
| (E)-6-(2,6,6-trimethyl-1-cyclohexen-1- | 0-10 |
| yl)ethenyl)-2-naphthalenecarboxylic acid | |
| (E,E,E)-7-(2,3-dihydro-1,1,3,3- | 0-10 |
| tetramethyl-1H-inden-5-yl)-3-methyl- | |
| 2,4,6-octatrenoic acid | |
| (E)-4-[2-(2,3-dihydro-1,1,3,3-tetramethyl- | 0-10 |
| 1H-inden-5-yl)-1- | |
| propenyl)benzoic acid TTNPB | |
| (E)-4-[2-(5,6,7,8-tetrahydro-5,5,8,8- | 0-10 |
| tetramethyl-2-naphthalenyl-1- | |
| propenyl]benzoic acid | |
| (E)-4-[2-(5,6,7,8-tetrahydro-3-methyl- | 0-10 |
| 5,5,8,8-tetramethyl-2-naphthalenyl-1- | |
| propenyl] benzoic acid | |
| (E)-1,2,3,4-tetrahydro-1,1,4,4,-tetramethyl- | 0-10 |
| 6-(1-methyl-2- | |
| phenylethenyl)naphthalene | |
| 6-(1,2,3,4-tetrahydro-1,1,4,4-tetramethyl- | 0-10 |
| 6-naphthyl)2-naphthalene- | |
| carboxylic acid | |
| (E)-6-[2-[4-(ethylsulfonyl)phenyl]-1- | 0-10 |
| methylethenyl]-1,2,3,4-tetrahydro- | |
| 1,1,4,4,-tetramethylnaphthalene | |
| 4-[(5,6,7,8-tetrahydro-5,5,8,8- | 0-10 |
| tetramethyl-2- | |
| naphthalenyl)ethynyl]benzoic acid | |
| (E)-2-(1,1,4,4-tetramethyl-1,2,3,4- | 0-10 |
| tetrahydronaphth-7-yl)-]-i-[4-tetrazol-5- | |
| yl)phenyl)-1-propene | |
| (E)-4-[2-(5,6,7,8-tetrahydro-7-hydroxy- | 0-10 |
| 5,5,8,8-tetramethyl-2-naphthalenyl)-1- | |
| propenyl]benzyl alcohol | |

### Enumerated Embodiments

[1.] The present invention provides a method of treating a skin disorder in a mammal, the method includes administering to the mammal a topical composition that includes an effective and nontoxic amount of xanthophylls.

[2.] The present invention also provides the method of embodiment 1, wherein the skin disorder is selected from the group of acne, eczema, psoriasis, rosacea, skin cancer, skin burns, skin allergies, congenital skin disorders, acantholysis, acanthosis, acanthosis nigricans, dermatosis, disease, erythroderma, furunculosis, impetigo, jungle rot, keratoderma, keratodermia, keratonosis, keratosis, keratosis nigricans, leukoderma, lichen, livedo, lupus, melanism, melanosis, molluscum, necrobiosis lipoidica, necrobiosis lipoidica diabeticorum, pemphigus, prurigo, rhagades, Saint Anthony's fire, seborrhea, vitiligo, xanthoma, xanthosis, Psoriatic arthritis, Reiter's syndrome, Guttate psoriasis, Dyshidriotic eczema, Acute and chronic graft versus host disease, Systemic sclerosis, Morphea, Spongiotic dermatitis, Allergic dermatitis, Nummular eczema, Pityriasis rosacea, Pityriasis rubra pilaris, Pemphigus erythematosus, Pemphigus vulgaris, Lichenoid keratosis, Lichenoid nitidus, Lichen planus, Lichenoid dermatitis, Seborrheic dermatitis, Autosensitization dermatitis, Dermatitis herpetiformis, and Eosinophilic dermatitis.

[3.] The present invention also provides the method of embodiment 1 wherein the skin disorder is a chronic skin disorder.

[4.] The present invention also provides a method for retarding or reversing the loss of collagen fibers, abnormal changes in elastic fibers, or deterioration of small blood vessels in sundamaged mammalian skin, the method comprising administering to the mammal a topical composition comprising an effective and nontoxic amount of xanthophylls.

[5.] The present invention also provides a method for exfoliating the skin surface of a mammal, the method comprising administering to the skin surface of a mammal in need thereof a topical composition comprising an effective and nontoxic amount of xanthophylls.

[6.] The present invention also provides a method for treating acne or a pimple in a mammal, the method comprising administering to a mammal in need thereof a topical composition comprising an effective and nontoxic amount of xanthophylls.

[7.] The present invention also provides the method of any one of embodiments 1-6, wherein the skin surface of the mammal is the face, neck, shoulder, chest, back, or any combination thereof.

[8.] The present invention also provides the method of any one of embodiments 1-7, wherein the mammal is a human.

[9.] The present invention also provides the method of any one of embodiments 1-8, wherein the mammalian skin is human facial skin.

[10.] The present invention also provides the method of any one of embodiments 1-9, wherein the xanthophylls exist in a vehicle that is a cream.

[11.] The present invention also provides the method of any one of embodiments 1-9, wherein the xanthophylls exist in a vehicle that is a ointment.

[12.] The present invention also provides the method of any one of embodiments 1-9, wherein the xanthophylls exist in a vehicle that is a lotion.

[13.] The present invention also provides the method of any one of embodiments 1-9, wherein the xanthophylls exist in a vehicle that is a gel.

[14.] The present invention also provides the method of any one of embodiments 1-9, wherein the xanthophylls exist in a vehicle that is a an emollient.

[15.] The present invention also provides the method of any one of embodiments 1-14, wherein the xanthophylls include lutein, zeaxanthin, capsorubin, capsanthin, astaxanthin, canthaxanthin, or any combination thereof.

[16.] The present invention also provides the method of any one of embodiments 1-14, wherein the xanthophylls include lutein and zeaxanthin.

[17.] The present invention also provides the method of any one of embodiments 1-14, wherein the xanthophylls include lutein and zeaxanthin, wherein the lutein is provided in the non-esterified form.

[18.] The present invention also provides the method of any one of embodiments 1-14, wherein the xanthophylls include lutein and zeaxanthin, wherein the lutein is provided as trans-lutein.

[19.] The present invention also provides the method of any one of embodiments 1-14, wherein the xanthophylls include lutein and zeaxanthin, wherein the lutein is provided as trans-lutein that is at least about 50 wt. % pure.

[20.] The present invention also provides the method of any one of embodiments 1-14, wherein the xanthophylls include lutein and zeaxanthin, wherein the lutein is provided as trans-lutein that is about 50 wt. % to about 90 wt. % pure.

[21.] The present invention also provides the method of any one of embodiments 1-20, wherein the topical composition further includes a skin conditioner.

[22.] The present invention also provides the method of any one of embodiments 1-21, wherein the topical composition further includes a skin conditioner selected from the group of calamine, aloe, lanolin, glycerin, Vitamin E, Vitamin E acetate, famesol, glycyrrhetinic acid, and combinations thereof.

[23.] The present invention also provides the method of any one of embodiments 1-22, wherein the topical composition further includes Vitamin A.

[24.] The present invention also provides the method of any one of embodiments 1-23, wherein the topical composition further includes one or more antimicrobial agents.

[25.] The present invention also provides the method of any one of embodiments 1-24, wherein the topical composition further includes an antimicrobial agent selected from the group of a β-lactam compound, an aminoglycoside, an antifungal agent, and combinations thereof.

[26.] The present invention also provides the method of any one of embodiments 1-25, wherein the topical composition further includes an antimicrobial agent selected from the group of erythromycin, tetracycline, clindamycin, and cephalosporin.

[27.] The present invention also provides the method of any one of embodiments 1-26, wherein the topical composition further includes one or more antiseptic agents.

[28.] The present invention also provides the method of any one of embodiments 1-27, wherein the topical composition further includes an antiseptic agent selected from the group of triclosan, phenoxy isopropanol, chlorhexidine gluconate, povidone iodine, and combinations thereof.

[29.] The present invention also provides the method of any one of embodiments 1-28, wherein the topical composition further includes a penetration enhancer.

[30.] The present invention also provides the method of any one of embodiments 1-29, wherein the topical composition further includes a penetration enhancer selected from the group of diethylene glycol monoethyl ether (transcutol), dimethyl sulfoxide (DMSO), propyleneglycol, ionic surfactants, non-ionic surfactants, anionic surfactants, isopropyl myristate (IPM), calcipotriene, detergents, emollients, chelators (e.g., calcium chelators such as EDTA, EGTA), Loramide DEA, Ethoxydiglycol, NMP, Triacetin, Propylene Glycol, Benzyl Alcohol, Sodium Laureth Sulfate, Dimethyl Isosorbide, Isopropyl Myristate, Olive Squalane, Medium Chain Triglyceride Oil (MCT Oil), Menthol, Isopropyl Palmitate, Isopropyl Isostearate, Propylene Glycol Monostearate, Lecithin, Diisopropyl Adipate, Diethyl Sebacate, Oleic Acid, Ethyl Oleate, Urea, Glyceryl Oleate, Caprylic/Capric Triglyceride, Propylene Glycol Dicaprylate/Dicaprate, Laureth-4, Oleth-2, Oleth-20, Propylene Carbonate, Nonoxynol-9,2-n-nonyl-1,3-dioxolane, C₇ to C₁₄-hydrocarbyl substituted 1,3-dioxolane, 1,3-dioxane, or acetal, Nonoxynol-15, and combinations thereof.

[31.] The present invention also provides the method of any one of embodiments 1-30, wherein the topical composition further includes a penetration enhancer present in the composition in about 0.1 wt. % to about 20.0 wt. %

[32.] The present invention also provides the method of any one of embodiments 1-31, wherein the topical composition further includes a keratolytic agent.

[33.] The present invention also provides the method of any one of embodiments 1-32, wherein the topical composition further includes a keratolytic agent selected from the group of alcloxa, resorcinol, and combinations thereof.

[34.] The present invention also provides the method of any one of embodiments 1-33, wherein the topical composition further includes Vitamin A, all-trans retinoic acid, or a combination thereof.

[35.] The present invention also provides the method of any one of embodiments 1-34, wherein the topical composition further includes a topical acne drug.

[36.] The present invention also provides the method of any one of embodiments 1-35, wherein the topical composition further includes a topical acne drug selected from the group of salicylic acid, resorcinol, resorcinol acetate, benzoyl peroxide, sulfur, retinol, retinoic acid, citric acid, an alpha hydroxy acid, retinal, a pharmaceutically acceptable salt thereof, and combinations thereof

[37.] The present invention also provides the method of any one of embodiments 1-36, wherein the topical composition further includes salicylic acid, or a pharmaceutically acceptable salt thereof, present in about 0.5 wt. % to about 2.0 wt. % of the composition.

[38.] The present invention also provides the method of any one of embodiments 1-37, wherein the topical composition further includes sulfur, present in about 3.0 wt. % to about 10.0 wt. % of the composition.

[39.] The present invention also provides the method of any one of embodiments 1-38, wherein the topical composition further includes a retinoid.

[40.] The present invention also provides the method of any one of embodiments 1-39, wherein the topical composition further includes a retinoid selected from the group of retinoic acids, retinoic acid derivatives and stereoisomers thereof.

[41.] The present invention also provides the method of any one of embodiments 1-40, wherein the topical composition further includes 13-cis-retinoic acid, 13-cis-retinoic acid derivatives and thereof

[42.] The present invention also provides the method of any one of embodiments 1-41, wherein the topical composition further includes a retinoid selected from the group of (E)-4-[4-methyl-6-(2,6,6-trimethyl-1-cyclohexen-1-yl)-] benzoic acid; 4-[(5,6,7,8-tetrahydro-5,5,8,8-tetramethyl-2-naphthalenyl)ethylnyl]-benzoic acid; and (E)-4-[2-(5,6,7,8-tetrahydro-7-hydroxy-5,5,8,8-tetramethyl-2-naphthalenyl-1-propenyl] benzyl alcohol.

[43.] The present invention also provides the method of any one of embodiments 1-42, wherein the topical composition further includes a retinoid selected from the group of 13-cis-retinoic acid; (all-E)-9-(4-methoxy-2,3,6-trimethylphenyl)-3,7-dimethyl-2,4,6,8-nonatetraenoic acid ethyl ester; (all-E)-9-(4-methoxy-2,3,6,-trimethylphenyl)-3,7-dimethyl-2,4,6,8,-nonatetraenoic acid; N-ethyl-9-(4-methoxy-2,3,6-trimethylphenyl)-3,7-dimethyl-2,4,6,8-nonatetraenamide; (E,E)-9-(2,6-dichloro-4-methoxy-3-methylphenyl)-3,7-dimethyl-2,4,6,8-nonatetraenoic acid ethyl ester; 7,8-didehydroretinoic acid; (E,E)-4-[2-methyl-4-(2,6,6-trimethyl-1-cyclohexen-1-yl)-1,3-butadienyl]benzoic acid; (all-E)-3,7-dimethyl-(3-thienyl)-2,4,6,8-nonatetraconic acid; (E,E,E)-3-methyl-7-(5,6,7,8-tetrahydro-5,5,8,8-tetramethyl-2-naphthalenyl)-2,4,6-octatrienoic acid; (E)-6-[2-(2,6,6-trimethyl-1-cyclohexen-1-yl)ethenyl]-2-naphthalenecarboxylic acid; (E,E,E)-7-(2,3-dihydro-1,1,3,3-tetramethyl-1H-inden-5-yl)-3-methyl-2,4,6-octatrienoic acid; (E)-4-[2-(2,3-dihydro-1,1,3,3-tetramethyl-1H-inden-5-yl)-1-propenyl]benzoic acid; TTNPB (E)-4-[2-(5,6,7,8-tetrahydro-5,5,8,8-tetramethyl-2-naphthalenyl-1propenyl] benzoic acid; (E)-4-[2-(5,6,7,8-tetrahydro-3-methyl-5,5,8,8-tetramethyl-2-naphthalenyl)-1-propenyl]benzoic acid; (E)-1,2,3,4-tetrahydro-1,1,4,4-tetramethyl-6-(1-methyl-2phenylethenyl) naphthalene; 6-(1,2,3,4-tetrahydro-1,1,4,4-tetra-methyl-6-naphthyl)-2-naphthalene-carboxylic acid; [(E)-6-[2-(4-(ethylsulfonyl)phenyl]-1-methylethenyl]-1,2,3,4-tetrahydro-1,2,3,4-tetrahydronaphth-7-yl)-1-[4-tetrazol-5-yl)phenyl]-1-propene] (E)-2-(1,1,4,4-tetramethyl-1,2,3,4-tetrahydronaphth-7-yl)-1-(4-tetra zol-5-yl)phenyl)-1-propene.

The following illustrate representative pharmaceutical dosage forms, containing xanthophylls, for therapeutic, and/or prophylactic use in humans.

| | | |
|---|---|---|
| Component | Function | Amount (% w/w) |

Solution Formulation:

| | | |
|---|---|---|
| Xanthophylls | Active | 0.10-10.0 |
| Isopropyl Alcohol | Solvent | 35-55 |
| Propylene Glycol | Solvent | 1-15 |
| Hydroxypropyl Cellulose | Thickening agent | 0-5 |
| Phosphoric Acid | Acidifying Agent | pH = 7-9 |
| Dibasic sodium phosphate | Base | 0.01-1.5 |
| Menthol | Odorant | 0-1 |
| Purified Water | Diluent | 25-80 |

Gel Formulation:

| | | |
|---|---|---|
| Xanthophylls | Active | 0.10-10.0 |
| Propylene glycol | Solvent | 0.1-10 |
| Methylparaben | Preservative | 0.01-0.1 |
| Propylparaben | Preservative | 0.01-0.1 |
| Edetate Disodium | Chelating agent | 0.01-0.1 |
| Dibasic sodium phosphate | Basic Agent | 0.01-1.5 |
| Carbomer | Gelling Agent | 0.1-2 |
| Phosphoric Acid | Neutralizing Agent | QS pH 7-9 |
| Ethanol | Solvent | 0-75 |
| Purified Water | Solvent | 25-95 |

Cream Formulation:

| | | |
|---|---|---|
| Xanthophylls | Active | 0.10-10.0 |
| Sorbitol 70% Solution | Humectant | 1-3 |
| Emulsifying Wax | Cream Base | 5-25 |
| Glycerin | Emollient | 0-20 |
| Isopropyl Palmitate | Penetration | 1-10 |
| Beuzyl alcohol | Preservative | 0.1-0.5 |
| Edetate disodium | Chelating agent | 0.01-0.55 |
| Dibasic sodium phosphate | Basic Agent | 0.01-0.55 |
| Phosphoric Acid | Neutralizing Agent | pH 7-9 |
| Ceteth 20 | Surfactant | 0-5 |
| Mineral Oil | Emollient | 0-55 |
| Purified Water | Solvent | 50-80 |

Ointment Formulation:

| Excipient | Function | Amount (% w/w) |
|---|---|---|
| Xanthophylls | Active | 0.10-10.0 |
| Microcrystalline Wax | Ointment base | 1-15 |
| White Petrolatum | Ointment base | 55-99 |
| Tocopherol | Anti-oxidant | 0-0.5 |
| Steareth-2 | Surfactant | 1-10 |
| Propylene Glycol | Solvent | 1-10 |
| Edetate disodium | Chelating agent | 0.001-0.55 |
| Dibasic sodium phosphate | Basic Agent | 0.01-0.55 |
| Phosphoric Acid | Neutralizing Agent | pH 7-9 |
| Purified Water | Solvent | 1-10 |

All publications and patent applications cited herein are incorporated by reference to the same extent as if each individual publication or patent application was specifically and individually indicated to be incorporated by reference.

Although certain embodiments have been described in detail above, those having ordinary skill in the art will clearly understand that many modifications are possible in the embodiments without departing from the teachings thereof. All such modifications are intended to be encompassed within the enumerated embodiments of the invention.

1. A method for treating acne or a pimple in a human, the method comprises administering to a human in need thereof a topical composition comprising an effective and nontoxic amount of xanthophylls.

2. The method of claim 1, wherein the skin surface of the human is the face, neck, shoulder, chest, back, or any combination thereof.

3. The method of claim 1, wherein the xanthophylls exist in a vehicle that is a cream, ointment, lotion, gel or emollient.

4. The method of claim 1, wherein the xanthophylls comprise lutein and zeaxanthin.

5. The method of claim 1, wherein the xanthophylls comprise lutein and zeaxanthin, wherein the lutein is provided in the non-esterified form.

6. The method of claim 1, wherein the xanthophylls comprise lutein and zeaxanthin, wherein the lutein is provided as trans-lutein.

7. The method of claim 1, wherein the xanthophylls comprise lutein and zeaxanthin, wherein the lutein is provided as trans-lutein that is at least about 50 wt. % pure.

8. The method of claim 1, wherein the xanthophylls comprise lutein and zeaxanthin, wherein the lutein is provided as trans-lutein that is about 50 wt. % to about 90 wt. % pure.

9. The method of claim 1, wherein the topical composition further comprises a skin conditioner selected from the group of calamine, aloe, lanolin, glycerin, Vitamin E, Vitamin E acetate, famesol, glycyrrhetinic acid, and combinations thereof.

10. The method of claim 1, wherein the topical composition further comprises one or more antimicrobial agents.

11. The method of claim 1, wherein the topical composition further comprises an antimicrobial agent selected from the group of a β-lactam compound, an aminoglycoside, an antifungal agent, and combinations thereof.

12. The method of claim 1, wherein the topical composition further comprises an antimicrobial agent selected from the group of erythromycin, tetracycline, clindamycin, and cephalosporin.

13. The method of claim 1, wherein the topical composition further comprises an antiseptic agent selected from the group of triclosan, phenoxy isopropanol, chlorhexidine gluconate, povidone iodine, and combinations thereof.

14. The method of claim 1, wherein the topical composition further comprises a penetration enhancer selected from the group of diethylene glycol monoethyl ether (transcutol), dimethyl sulfoxide (DMSO), propyleneglycol, ionic surfactants, non-ionic surfactants, anionic surfactants, isopropyl myristate (IPM), calcipotriene, detergents, emollients, chelators (e.g., calcium chelators such as EDTA, EGTA), Loramide DEA, Ethoxydiglycol, NMP, Triacetin, Propylene Glycol, Benzyl Alcohol, Sodium Laureth Sulfate, Dimethyl Isosorbide, Isopropyl Myristate, Olive Squalane, Medium Chain Triglyceride Oil (MCT Oil), Menthol, Isopropyl Palmitate, Isopropyl Isostearate, Propylene Glycol Monostearate, Lecithin, Diisopropyl Adipate, Diethyl Sebacate, Oleic Acid, Ethyl Oleate, Urea, Glyceryl Oleate, Caprylic/Capric Triglyceride, Propylene Glycol Dicaprylate/Dicaprate, Laureth-4, Oleth-2, Oleth-20, Propylene Carbonate, Nonoxynol-9,2-n-nonyl-1,3-dioxolane, C₇ to C₁₄-hydrocarbyl substituted 1,3-dioxolane, 1,3-dioxane, or acetal, Nonoxynol-15, and combinations thereof.

15. The method of claim 1, wherein the topical composition further comprises a keratolytic agent selected from the group of alcloxa, resorcinol, and combinations thereof.

16. The method of claim 1, wherein the topical composition further comprises Vitamin A, all-trans retinoic acid, or a combination thereof.

17. The method of claim 1, wherein the topical composition further comprises a topical acne drug selected from the group of salicylic acid, resorcinol, resorcinol acetate, benzoyl peroxide, sulfur, retinol, retinoic acid, citric acid, an alpha hydroxy acid, retinal, a pharmaceutically acceptable salt thereof, and combinations thereof.

18. The method of claim 1, wherein the topical composition further comprises salicylic acid, or a pharmaceutically acceptable salt thereof, present in about 0.5 wt. % to about 2.0 wt. % of the composition.

19. The method of claim 1, wherein the topical composition further comprises sulfur, present in about 3.0 wt. % to about 10.0 wt. % of the composition.

20. The method of claim 1, wherein the topical composition further comprises a retinoid selected from the group of 13-cis-retinoic acid; (all-E)-9-(4-methoxy-2,3,6-trimethylphenyl)-3,7-dimethyl-2,4,6,8-nonatetraenoic acid ethyl ester; (all-E)-9-(4-methoxy-2,3,6,-trimethylphenyl)-3,7-dimethyl-2,4,6,8,-nonatetraenoic acid; N-ethyl-9-(4-methoxy-2,3,6-trimethylphenyl)-3,7-dimethyl-2,4,6,8-nonatetraenamide; (E,E)-9-(2,6-dichloro-4-methoxy-3-methylphenyl)-3,7-dimethyl-2,4,6,8-nonatetraenoic acid ethyl ester; 7,8-didehydroretinoic acid; (E,E)-4-[2-methyl-4-(2,6,6-trimethyl-1-cyclohexen-1-yl)-1,3-butadienyl]benzoic acid; (all-E)-3,7-dimethyl-(3-thienyl)-2,4,6,8-nonatetraconic acid; (E,E,E)-3-methyl-7-(5,6,7,8-tetrahydro-5,5,8,8-tetramethyl-2-naphthalenyl)-2,4,6-octatrienoic acid; (E)-6-[2-(2,6,6-trimethyl-1-cyclohexen-1-yl)ethenyl]-2-naphthalenecarboxylic acid; (E,E,E)-7-(2,3-dihydro-1,1,3,3-tetramethyl-1H-inden-5-yl)-3-methyl-2,4,6-octatrienoic acid; (E)-4-[2-(2,3-dihydro-1,1,3,3-tetramethyl-1H-inden-5-yl)-1-propenyl]benzoic acid; TTNPB (E)-4-[2-(5,6,7,8-tetrahydro-5,5,8,8-tetramethyl-2-naphthalenyl-1propenyl] 0benzoic acid; (E)-4-[2-(5,6,7,8-tetrahydro-3-methyl-5,5,8,8-tetramethyl-2-naphthalenyl)-1-propenyl]benzoic acid; (E)-1,2,3,4-tetrahydro-1,1,4,4-tetramethyl-6-(1-methyl-2phenylethenyl) naphthalene; 6-(1,2,3,4-tetrahydro-1,1,4,4-tetra-methyl-6-naphthyl)-2-naphthalene-carboxylic acid; [(E)-6-[2-(4-(ethylsulfonyl)phenyl]-1-methylethenyl]-1,2,3,4-tetrahydro-1,2,3,4-tetrahydronaphth-7-yl)-1-[4-tetrazol-5-yl)phenyl]-1-propene] (E)-2-(1,1,4,4-tetramethyl-1,2,3,4-tetrahydronaphth-7-yl)-1-(4-tetra zol-5-yl)phenyl)-1-propene.
